(19) United States
(12) Patent Application Publication
(10) Pub. No.: US 2022/0331195 A1
(43) Pub. Date: Oct. 20, 2022

(54) AIR MICROFLUIDICS AND AIR MINIFLUIDICS ENABLED ACTIVE COMPRESSION DEVICE, APPAREL, AND METHOD

(71) Applicants: Carolyn Liqing REN, Waterloo (CA); Run Ze GAO, Waterloo (CA)

(72) Inventors: Carolyn Liqing REN, Waterloo (CA); Run Ze GAO, Waterloo (CA)

(21) Appl. No.: 17/620,921

(22) PCT Filed: Jun. 24, 2020
(86) PCT No.: PCT/CA2020/050874

§ 371 Date: Dec. 20, 2021

Related U.S. Application Data
(60) Provisional application No. 62/865,565, filed on Jun. 24, 2019.

(30) Foreign Application Priority Data
Jun. 25, 2019 (CA) 3047880

(51) Int. Cl. A61H 9/00; B25J 9/00

(52) U.S. Cl. CPC A61H 9/0092; B25J 9/0006; A61H 2201/165; A61H 2201/1238; A61H 2230/625; A61H 2201/1409; A61H 2201/5056; A61H 2201/5007; A61H 2201/5058; A61H 2205/102

(57) ABSTRACT

Air microfluidics and minifluidics enabled active compression apparel enhances mobility and quality of life for individuals by minimizing risks of injuries, enhancing rehabilitation, and maximizing comfort. Balloon actuators, integrated with a garment, provide active compression and augmenting forces to anatomical portions of the human body. The balloon actuators are actuated by fluidic pressurization hardware. The air microfluidics and minifluidics system miniaturizes fluidic pressurization hardware and makes it wearable, ultra lightweight, and ultra formfitting. The air microfluidics and minifluidics system includes micro and mini channels of various lengths, cross-sectional areas, and functions via principles of equivalent hydraulic resistance allowing for fluidic transportation, passive delay in pressurization of the balloon actuators, and digital soft fluidic actuation where the compression force is based on the number of inflated balloon actuators instead of their pressure.

### FIELD OF THE INVENTION

The present invention relates generally to the field of assistive devices, compression apparel, and soft wearable robotics, and more specifically to active compression garments for rehabilitation, sports, recreation and increasing quality of life for its users. Even more specifically, the present invention uses air microfluidics and air minifluidics techniques to create fully untethered, ultra lightweight, ultra formfitting and aesthetically pleasing active compression apparel to enhance the user's mobility, relieve pain, and increase comfort.

### BACKGROUND OF THE INVENTION

Exoskeletons and rigid braces may have, by and large, occupied the realm of assistive devices and may have been categorized as “hard” assistive devices, which may have been either active or passive systems.

Soft wearable robotics may have been orthotics or prosthetics. The majority of soft wearable robotics may have used soft fluidic actuators to provide assistance to the human body. This assistance may typically have come in the form of an augmenting force or torque. The soft fluidic actuators may have been actuated by liquid or gas. Compared to traditional hydraulic and pneumatic actuators, soft fluidic actuators may be more lightweight, smaller in size, less bulky, more compliant, and/or cheaper to fabricate. Typically, soft wearable robots may have had multiple independently controlled fluidic chambers, and these fluidic chambers are usually controlled by valves to actively inflate and deflate. The timing and/or sequencing of these valves may have been typically determined by software programming. Another type of timing and sequencing may have been determined by adjusting the material property and thickness of the walls of each fluidic chamber. These types of fluidic actuators may have been useful in creating propulsion via torque around a joint or limb, but may not have been ideal for compression.

Although soft fluidic actuators may have been compliant and/or form-fitting, their control system still may have been bulky and/or large which may have made overall systems challenging to become untethered. Untethering the systems typically may have required reducing the number and/or shrinking the size of valves to reduce the weight of the control system. Shrinking the size of valves still may pose challenges of mechanical failures and/or increases to the cost of fabrication. Hence, it may have been unsustainable and/or challenging to scale up for commercial production. Reducing the number of solenoid valves might pose challenges of functionality and/or aesthetics of soft fluidic actuators.

### DESCRIPTION OF PRIOR ART

Patent reference WO 2015/102723 A2 may disclose a mechanically programmed soft fluidic actuator that may be configured to bend, linearly extend, contract, twist or combinations thereof with usage of a sleeve wrapped around part of the soft actuator.

Patent reference WO 2015/050853 A1 may disclose methods of using and/or making a soft composite fluidic actuator, potentially including an elastomeric layer, a strain limiting layer, and/or a radially constraining layer. All such layers may have been bonded together to form a bladder for holding pressurized fluid.

Patent reference U.S. Pat. No. 9,945,397 B2 may disclose systems and/or methods for actuating soft robotic actuators.

Patent reference U.S. Pat. No. 6,637,463 B1 may disclose methods and/or apparatus for controlling fluid flow through flow paths with pressure gradient fluid control. Passive fluid flow elements such as barriers may have allowed for fluid flow to be regulated.

Patent reference US 2018/0143091 A1 may disclose an artificial skin and/or elastic strain formed by filling channels in an elastic substrate material with a conductive liquid.

Patent reference WO 2013/033669 A2 may disclose an actively controlled wearable orthotic device and/or active modular elastomer sleeve for wearable orthotic devices. The orthotic device may be used for locomotion assistance, gait rehabilitation, and/or gait training.

Patent reference WO 2018/220596 A2 may disclose a soft portable wearable pneumatic interactive suit for communication and/or information transfer between users and/or machines. The interactive suit may include actuators, sensing elements, and/or a portable control device.

Patent reference EP 1 133 652 B1 may disclose a manifold system of removable components for distribution of fluids.

Patent reference U.S. Pat. No. 7,976,795 B2 may disclose a microfluidics system comprising a pneumatic manifold having apertures, and a chip manifold having channels disposed for routing pneumatic signals from respective apertures to valves in a microfluidic chip.

Patent reference U.S. Pat. No. 8,595,922 B2 may disclose a method for making flexible silicone cable system integrated with snap washers. The silicone cable system may transport fluid.

Patent reference US 2017/0128008 A1 may disclose a process and/or method for making flexible and/or wearable microfluidic channel structures and/or devices. The microfluidic channel structures and/or devices may be printed on textiles.

Patent reference U.S. Pat. No. 8,657,772 B2 may disclose a wearable device having feedback characteristics that may be integrated with a compliant article for providing a user with information regarding a range of motion parameters of a joint and/or to condition users to maintain proper joint orientations.

Patent reference WO 2018/222930 A1 may disclose textile actuators made out of a fluid bladder surrounded by a textile envelope that may be worn by a user for displacing a body segment of the user and/or may support and/or hold the body segment of the user in place.

Patent reference US 2017/0239821 A1 may disclose a soft robotic device with embedded sensors.

Patent reference US 2012/0238914 A1 may disclose an actively controlled orthotic device. The orthotic device may be applied to the wrist, elbow, torso, and/or another body part.

Patent reference US 2015/0148619 A1 may disclose a wearable system for monitoring biometric signals of a user.

Patent reference U.S. Pat. No. 6,296,020 B1 may disclose methods of controlling fluid flow through microchannels by use of passive valves or stopping means in the microchannels.

Patent reference U.S. Pat. No. 8,286,665 B2 may disclose multiplexed latching valves for microfluidic devices and/or processors that may be used to form pneumatic logic circuits.

Patent reference US 2010/0292706 A1 may disclose a modular, scalable, layerable balloon actuator and/or actuator array.

Patent reference U.S. Pat. No. 9,652,037 B2 may disclose a human-computer interface system having an exoskeleton that may be configured to apply a force to a body segment of the user.

The prior art canvassed above, however, may have suffered from one or more significant problems and/or shortcomings. For example, all this prior art may have suffered from one or more of the following problems: (a) lack of wearability regarding on-the-go usage; (b) inefficient and/or bulky actuation hardware; (c) using only constant passive compression methods; (d) unable to achieve sequential actuation and/or selective actuation of a plurality of balloon actuators; (e) inefficient large volume balloon actuators; and/or (f) poor user compliance, perhaps at least in part due to mechanical, electronics, and/or software shortcomings.

What may be needed is an air microfluidics and air minifluidics enabled active compression device, apparel, and/or method, or a wearable air microfluidics and minifluidics device, garment, and method for active compression.

It may be desirable to provide a device, garment, and/or method that uses novel air microfluidics and air minifluidics to miniaturize the control system of active compression apparels by replacing numerous electromechanical valves with one or more much more compact air microfluidic chips. The miniaturized control system may present better efficiency, less bulk, and/or novel sequential inflation and/or deflation, and/or selection methods for a plurality and/or an array of balloon actuators (i.e., otherwise known as air chambers or pneumatic bladders).

Additionally or instead, it may be desirable to provide a device, garment, and/or method that may preferably, and without being bound by theory or analogy, uses the theory of equivalent hydraulic resistance and/or an electrical circuit analogy, and/or otherwise minimizes the usage of numerous pressure sensors during the inflation/deflation of a plurality or an array of balloon actuators, which is coined “digital soft fluidic actuation” as the amount of compression can be controlled without knowing each individual balloon actuator's pressure.

Yet further, it may be advantageous to provide a device, garment, and/or method that uses an array of balloon actuators, which allow for better distribution of compression compared to a single large air chamber and/or allows for sequential inflation and/or deflation.

It may be desirable to provide a device, garment, and/or method that may find advantageous utility in association with one or more of the following applications: (i) Osteoarthritic knee unloading braces—unicompartmental and/or multicompartmental unloading and proprioception; (ii) Lymphedema treatment; (iii) Deep vein thrombosis treatment; (iv) Dynamic prosthetic socket liners; (v) Joint stabilization sleeves—post-surgery or prophylactic; (vi) Neck and back massages; (vii) Repetitive strain injury treatment, including, for example, muscles, ligaments, tendons, and/or carpal tunnel; (viii) Workout massages—warmup and lactic acid removal; and/or (ix) Athleisure apparel, i.e. yoga pants, compression clothing, and sports bras—movement/posture synchronized force tactile sensation and haptics during a workout.

It may be an object according to one aspect of the invention to provide an air microfluidics and air minifluidics enabled active compression device, apparel, and/or method.

It may be an object according to one aspect of the invention to provide a wearable air microfluidics and minifluidics device, garment, and method for active compression.

It is an object of the present invention to obviate or mitigate one or more disadvantages and/or shortcomings associated with the prior art, to meet or provide for one or more needs and/or advantages, and/or to achieve one or more objects of the invention—one or more of which may preferably be readily appreciable by and/or suggested to those skilled in the art in view of the teachings and/or disclosures hereof.

### SUMMARY OF THE INVENTION

According to the invention, there is disclosed a wearable air microfluidics and minifluidics device, for use with one or more garments worn by a user. The device preferably includes balloon actuators, an air channel module, a pneumatic module, one or more sensors, and a control module. The balloon actuators are preferably integrated with the garments, and apply one or more predetermined forces to one or more anatomical portions of the user's body when inflated with gas. The forces preferably include active compression and/or augmenting forces. (Persons having ordinary skill in the art should readily appreciate, in view of the disclosures herein, that the term “balloon actuators” may be broad enough to reasonably encompass anything that, through inflation and/or deflation, can apply the forces to the anatomical portions of the user's body—including, for example, tube actuators among other things.) The air channel module preferably includes one or more small-scale air channels in fluid communication with the balloon actuators. The small-scale air channels preferably include air micro channels and/or air mini channels. The pneumatic module preferably is in fluid communication via the small-scale air channels with the balloon actuators. The pneumatic module, when activated, preferably induces flow of the gas under pressure, through the small-scale air channels, to the balloon actuators. The sensors preferably are integrated with the garments, and generate signals based on biometric data and/or user motion detected at the garment. Preferably, the control module selectively, depending upon the signals from the sensors, activates the pneumatic module to inflate and deflate the balloon actuators, to apply the predetermined forces to the anatomical portions of the user's body, based on the biometric data and/or user motion.

According to an aspect of a preferred embodiment of the invention, the air channel module, the pneumatic module, and/or the control module may preferably, but need not necessarily, be securely attached to the garments.

According to an aspect of a preferred embodiment of the invention, the air channel module may preferably, but need not necessarily, be configured to use (and/or utilize) equivalent hydraulic resistance and/or to induce passive delays in pressurization and/or depressurization of the balloon actuators.

According to an aspect of a preferred embodiment of the invention, the aforesaid passive delays in the aforesaid pressurization and/or depressurization, preferably via the small-scale air channels, may preferably (but need not necessarily) enable digital soft fluidic actuation of the balloon actuators.

According to an aspect of a preferred embodiment of the invention, the small-scale air channels may preferably, but need not necessarily, be configured to have different cross-sectional areas, cross-sectional shapes, channel lengths, channel characteristic dimensions, and/or channel routes.

According to an aspect of a preferred embodiment of the invention, the small-scale air channels may preferably, but need not necessarily, be configured to be connected in series and/or parallel fluid communication with the balloon actuators.

According to an aspect of a preferred embodiment of the invention, at least a portion of the air channel module may preferably, but need not necessarily, be integrated with the garments in selectively removable relation.

According to an aspect of a preferred embodiment of the invention, some of the small-scale air channels may preferably, but need not necessarily, be selectively blocked off, and the remainder of the small-scale air channels may preferably (but need not necessarily) remain, in fluid communication with the balloon actuators.

According to an aspect of a preferred embodiment of the invention, the small-scale air channels may preferably, but need not necessarily, be combined into a network. Each respective one of the small-scale air channels may preferably, but need not necessarily, be elastic, flexible, and/or rigid.

According to an aspect of a preferred embodiment of the invention, the air channel module may preferably, but need not necessarily, also include: (a) one or more air microfluidics chips, and/or (b) an elastic mini channel network that may preferably, but need not necessarily, be integrated with the garments. The small-scale air channels may preferably, but need not necessarily, be embodied in the air microfluidics chips and/or in the elastic mini channel network. At least some of the small-scale air channels embodied in the elastic mini channel network may preferably, but need not necessarily, be the aforesaid air mini channels and may preferably, but need not necessarily, be elastic.

According to an aspect of a preferred embodiment of the invention, the air channel module may preferably, but need not necessarily, include an air microfluidics socket. The air microfluidics socket may preferably, but need not necessarily, be adapted to receive at least a first selected one of the air microfluidics chips in fluid communication with the elastic mini channel network.

According to an aspect of a preferred embodiment of the invention, the air microfluidics socket may preferably, but need not necessarily, receive the aforesaid first selected one of the air microfluidics chips in selectively removable relation. The air microfluidics socket may preferably, but need not necessarily, be further adapted to alternately receive a second selected one of the air microfluidics chips in fluid communication with the elastic mini channel network.

According to an aspect of a preferred embodiment of the invention, the first selected one and the second selected one of the air microfluidics chips may preferably, but need not necessarily, block off different sets of the small-scale air channels in fluid communication with the balloon actuators.

According to an aspect of a preferred embodiment of the invention, the balloon actuators may preferably, but need not necessarily, be elastic and/or flexible. (Persons having ordinary skill in the art should readily appreciate, in view of the disclosures herein, that the term “elastic” may be broad enough to reasonably encompass various forms of elasticity—for example, hyper-elastic and/or hyper-elasticity, among others.)

According to an aspect of a preferred embodiment of the invention, the balloon actuators may preferably, but need not necessarily, have a spherical shape, an elongated cylindrical shape, a donut shape, and/or an irregular shape.

According to an aspect of a preferred embodiment of the invention, the pneumatic module may preferably, but need not necessarily, include one or more pneumatic mini channels and/or pneumatic micro channels in fluid communication with the air channel module.

According to an aspect of a preferred embodiment of the invention, at least a portion of the pneumatic module may preferably, but need not necessarily, be integrated with the garments in selectively removable relation.

According to an aspect of a preferred embodiment of the invention, the pneumatic module may preferably, but need not necessarily, draw gas from the external environment.

According to an aspect of a preferred embodiment of the invention, the pneumatic module may preferably, but need not necessarily, include a fluidic reservoir. The pneumatic module may preferably, but need not necessarily, draw gas from the fluidic reservoir.

According to an aspect of a preferred embodiment of the invention, the pneumatic module may preferably, but need not necessarily, include an air filter.

According to an aspect of a preferred embodiment of the invention, the pneumatic module may preferably, but need not necessarily, include a mini/micro air pump and/or one or more mini/micro valves, integrated with the garments, in fluid communication with the small-scale air channels.

According to an aspect of a preferred embodiment of the invention, the control module may preferably, but need not necessarily, operatively execute an actuation subroutine. The actuation subroutine, in selectively activating the pneumatic module as aforesaid, may preferably, but need not necessarily, control the mini/micro air pump and/or the mini/micro valves.

According to an aspect of a preferred embodiment of the invention, the control module may preferably, but need not necessarily, operatively execute a sensor fusion subroutine. The sensor fusion subroutine may preferably, but need not necessarily, reconcile and/or combine the signals from the sensors into a substantially complete dataset of the biometric data and/or user motion that was detected at the garment.

According to an aspect of a preferred embodiment of the invention, the control module may preferably, but need not necessarily, operatively execute an artificial neural network subroutine, preferably to determine user motion patterns of the user's body and/or said anatomical portions of the user's body.

According to an aspect of a preferred embodiment of the invention, the control module may preferably, but need not necessarily, include physical hardware. At least some of the physical hardware may preferably, but need not necessarily, be integrated onboard the garments.

According to an aspect of a preferred embodiment of the invention, the device may preferably, but need not necessarily, be adapted for use with a portable computing device that is preferably off-board the garments. The control module may preferably, but need not necessarily, include one or more software components that, at least partially, are operatively executed and/or reside on the portable computing device.

According to an aspect of a preferred embodiment of the invention, the software components may preferably, but need not necessarily, enable the user, at least partially, to manually control selective activation of the pneumatic module as aforesaid, to input predetermined settings for automated control of the pneumatic module and/or the balloon actuators, to track performance and information regarding the pneumatic module and/or the balloon actuators, and/or to update the software components.

According to an aspect of a preferred embodiment of the invention, the device may preferably, but need not necessarily, also include one or more containers that at least partially contain the pneumatic module and/or the control module.

According to an aspect of a preferred embodiment of the invention, the containers may preferably, but need not necessarily, be integrated with the garments in selectively removable relation.

According to an aspect of a preferred embodiment of the invention, the containers may preferably, but need not necessarily, be elastic, flexible, and/or rigid.

According to an aspect of a preferred embodiment of the invention, the device may preferably, but need not necessarily, also include at least one electrical power module that preferably electrically powers the pneumatic module, the sensors, and/or the control module.

According to an aspect of a preferred embodiment of the invention, the electrical power module may preferably, but need not necessarily, include a battery and/or a transmission system that preferably provides electrical power to the pneumatic module, the sensors, and/or the control module.

According to an aspect of a preferred embodiment of the invention, the battery may preferably, but need not necessarily, be rechargeable and/or replaceable.

According to an aspect of a preferred embodiment of the invention, the device may preferably, but need not necessarily, be adapted for use with air as the gas.

According to an aspect of a preferred embodiment of the invention, the device may preferably, but need not necessarily, be adapted for use with garments which have an outer garment layer. Each of the balloon actuators may preferably, but need not necessarily, be positioned between the outer garment layer and the user's skin. According to an aspect of a preferred embodiment of the invention, the device may preferably, but need not necessarily, be adapted for use with garments which also have an inner garment layer that contacts the user's skin. Each of the balloon actuators may preferably, but need not necessarily, be positioned between the outer garment layer and the inner garment layer.

According to the invention, there is also disclosed an air microfluidics and minifluidics garment, adapted to be worn by a user. The garment preferably also includes one or more outer garment layers. The garment preferably also includes the aforesaid balloon actuators, air channel module, pneumatic module, sensors, electrical power module, and/or control module. According to an aspect of a preferred embodiment of the invention, the garment may preferably, but need not necessarily, also include at least one inner garment layer that contacts the user's skin. The balloon actuators may preferably, but need not necessarily, be positioned between the outer garment layers and the inner garment layer. The air channel module, the pneumatic module, the sensors, and the control module may preferably, but need not necessarily, be securely attached to the outer garment layers and/or to the inner garment layer.

According to an aspect of a preferred embodiment of the invention, the outer garment layers may preferably, but need not necessarily, be overlaid on top of each other, be selectively attachable at predetermined locations on the inner garment layer, and/or be selectively detachable from said predetermined locations on the inner garment layer.

According to an aspect of a preferred embodiment of the invention, a first one of the outer garment layers may preferably, but need not necessarily, be selectively attachable at, and/or detachable from, predetermined locations on a second one of the outer garment layers.

According to an aspect of a preferred embodiment of the invention, the outer garment layers may preferably, but need not necessarily, limit an inflatable size of the balloon actuators and/or direct the predetermined forces towards the user's body preferably when the balloon actuators are inflated.

According to the invention, there is also disclosed an air microfluidics and minifluidics method for applying one or more predetermined forces to one or more anatomical portions of a user's body. The method preferably includes a detection step, a control step, an air channel step, and/or a balloon actuation step. In the detection step, one or more sensors are preferably integrated with garments that are adapted to be worn by a user. The sensors are preferably used to generate signals based on biometric data and/or user motion detected at the garment. In the control step, a control module is preferably used to selectively, depending upon the signals from the sensors, activate a pneumatic module to induce flow of gas under pressure, through small-scale air channels of an air channel module. In the air channel step, the small-scale air channels of the air channel module are preferably used, in fluid communication with balloon actuators, to convey the flow of the gas under pressure to the balloon actuators. Before the air channel step, the small-scale air channels are preferably provided in the form of air micro channels and/or air mini channels. In the balloon actuation step, the balloon actuators are preferably integrated with the garments, and inflated and deflated to apply the predetermined forces to the anatomical portions of the user's body, preferably based on the biometric data and/or user motion. The forces preferably include active compression and/or augmenting forces.

According to an aspect of a preferred embodiment of the invention, in the air channel step, the air channel module may preferably, but need not necessarily, use equivalent hydraulic resistance and/or induce passive delays in pressurization and/or depressurization of the balloon actuators in the balloon actuation step.

According to an aspect of a preferred embodiment of the invention, in the balloon actuation step, said passive delays in said pressurization and/or depressurization, via the small-scale air channels, may preferably, but need not necessarily, enable digital soft fluidic actuation of the balloon actuators.

According to an aspect of a preferred embodiment of the invention, in the air channel step, a first selected air microfluidics chip may preferably, but need not necessarily, be provided in an air microfluidics socket in selectively removable relation. The air microfluidics socket may preferably, but need not necessarily, be adapted to alternately receive a second selected air microfluidics chip, in fluid communication with the small-scale air channels.

According to an aspect of a preferred embodiment of the invention, in the air channel step, the first selected air microfluidics chip and the second selected air microfluidics chip may preferably, but need not necessarily, block off different sets of the small-scale air channels in fluid communication with the balloon actuators.

According to an aspect of a preferred embodiment of the invention, in the control step, the control module may preferably, but need not necessarily, operatively execute an actuation subroutine that, in selectively activating the pneumatic module as aforesaid, preferably controls a mini/micro air pump and one or more mini/micro valves of the pneumatic module that are preferably integrated with the garments, in fluid communication with the small-scale air channels.

According to an aspect of a preferred embodiment of the invention, in the control step, one or more software components may preferably (but need not necessarily) be, at least partially, operatively executed on a portable computing device that is off-board the garments. The software components may preferably, but need not necessarily, enable the user, at least partially, to manually control selective activation of the pneumatic module as aforesaid, to input predetermined settings for automated control of the pneumatic module and/or the balloon actuators, to track performance and information regarding the pneumatic module and/or the balloon actuators, and/or to update the software components.

According to an aspect of a preferred embodiment of the invention, in the balloon actuation step, each of the balloon actuators may preferably, but need not necessarily, be positioned between outer garment layers of the garments, and an inner garment layer that contacts the user's skin.

According to an aspect of a preferred embodiment of the invention, prior to the balloon actuation step, the outer garment layers may preferably, but need not necessarily, be overlaid on top of each other, be selectively attached at predetermined locations on the inner garment layer, and/or be selectively detached from said predetermined locations on the inner garment layer.

According to an aspect of a preferred embodiment of the invention, in the balloon actuation step, the outer garment layers may preferably, but need not necessarily, limit an inflatable size of the balloon actuators and/or direct the predetermined forces towards the user's body.

According to the invention, there is also disclosed an exemplary air microfluidics and/or air minifluidics enabled active compression garment. Some of its system integration, fabrication, and/or applications may be described and/or illustrated below. Preferred embodiments, as well as various alternative embodiments of the systems, may also be described and/or illustrated below.

Perhaps at least part of this invention may lie in the junction point of soft wearable robotics, compression apparel, and/or microfluidics and/or minifluidics technology. One central and/or key distinguishing factor between this invention/improvement and prior arts may be in the use, methodology, and/or implementation of this air microfluidics and/or air minifluidics system's concept of equivalent hydraulic resistance to induce delay in flow, allowing for passive programming of the system using fluidic channels' resistance, perhaps instead of the thickness of the channel walls and/or material properties and/or valves. Also, the method of system integration of air microfluidics and/or air minifluidics channels within garments to achieve ultra formfitting, ultra lightweight, fully untethered, and/or increased efficiency may be among novel features disclosed and/or taught according to the present invention. Due to a low amount of mechanical moving parts, the system may be very robust, sturdy, and/or reliable. Also, the use of digital soft fluidic actuation methods may allow for minimal use of pressure sensors. Digital soft fluidic actuation may use the concept of paths of the least resistance, as the pressure in one balloon actuator reaches the predefined pressure, the fluidic flow is forced into other balloon actuators connected in parallel. When used in conjunction with the air microfluidics and air minifluidics system, the amount of compression can be controlled without the need of knowing each individual chamber's pressure.

Some advantages of this system, as compared to passive compression garments may include its abilities to tailor the level and/or location of compression on demand. Compared to active compression devices, the air microfluidics and/or air minifluidics implementations may allow for better aesthetics, better quality of compression, lowered fabrication cost, and/or complete washability. Compared to soft wearable robots, this system differs in its usage and actuation implementation. Another distinguishing factor is that the systems described herein are primarily used for actively compressing an anatomical portion of the human body instead of directly augmenting its movements. This compression can be used for massaging, minimizing the risk of injury, better proprioception, rehabilitation, as well as everyday comfort.

In any of the embodiments described herein, the pneumatic power may be provided by at least one mini air pump or pneumatic compressor.

In certain embodiments described herein, an air filter may be incorporated into the pneumatic power source to filter out dust, moisture, and any unwanted elements that could damage the internals of the systems.

In any of the embodiments described herein, at least one balloon actuator of any shape made out of plastic membrane or elastomer membrane or both is sandwiched between one or multiple outer strain limiting fabrics and an inner human skin contact fabric. These fabrics can be made out of any knitting pattern and material.

In any of the embodiments described herein, at least one mini or micro solenoid valve may be used to control the pneumatic power source.

In any of the embodiments described herein, at least one minifluidics channel, tubing, or channel network may be embedded in garment.

In certain embodiments described herein, at least one air microfluidics channel may be embedded in garment.

In certain embodiments described herein, at least one air microfluidics channel may be embedded within at least one detachable air microfluidics chip that may be attached to the garment via an air microfluidics socket embedded in garment.

In any of the embodiments described herein, the kinematics information of the human joints, limbs, and any body parts or the whole body may be captured by IMU (inertial measurements units) sensors or any appropriate sensors.

In some embodiments described herein, the electromyography information of human muscles may be measured and used in actuating the balloon actuators.

In any of the embodiments described herein, the signals from the sensors may be processed through a software algorithm to increase the signal to noise ratio and to determine the movement of the anatomical portion of the human body and activities of the human body in real time. Afterwards, the signals from the sensors are passed through an actuation algorithm to control the members within the pneumatic control container.

In some embodiments described herein, the user may calibrate and input the desired actuation levels through an application software on a mobile computing device.

In some embodiments described herein, the software algorithm may calibrate the actuation levels based on a deep artificial neural network.

In some embodiments described herein, sensor fusion algorithms may be used to combine and process the information from multiple sensors.

In some embodiments described herein, the pneumatic control container comprises at least one mini valve, at least one mini air pump, at least one mini tubing integrated with garment, at least one fluid reservoir and at least one air filter.

In some embodiments described herein, the sensors may transmit signals to the control center via physical wires or wireless transmission methods.

In an exemplary application, the systems described herein are used to actively increase knee stabilization, proprioception, bodily fluid circulation through tailored compression, ease pain caused by musculoskeletal injuries through tailored active compressional massages and mechanotherapy. The said tailored active compressional effect can also be used to minimize the injuries at and around the knees.

In a preferred embodiment of the proposed system, the pneumatic control container is attached to the apparel by mechanical or magnetic means and can be detached. The air minifluidics channel network conduit is fully integrated with the apparel. The air microfluidics chip is attached to the air microfluidics socket, which is integrated with the apparel, via mechanical or magnetic means and can be detached. The air microfluidics chip passively induces programmed delays in the pressurization of balloon actuators via the concept of equivalent hydraulic resistance, and the air microfluidics chip is also a selection manifold, allowing only certain balloon actuators to pressurize while fully blocking others. A single pressure sensor is used within the pneumatic control container to provide pressure feedback of the entire system. A set of IMU sensors measure the kinematics of the anatomical portion of the human body where the balloon actuators are intended for. A set of electromyography sensors measure the electrical signals of the muscles associated with the anatomical portion of the human body where the balloon actuators are intended for. The signals of the IMU and EMG sensors are sent to the control center, which is integrated with the apparel. The IMU and EMG sensors are first processed to improve the signal to noise ratio, then passed through sensor fusion algorithm and deep artificial neural network, and formed into control signals for the actuation algorithm that actuates the mini pumps and mini solenoid valves within the pneumatic control container. The control algorithms are tailored for each user through user-based calibration or inputs via an application software on a mobile computing device.

Other advantages, features and characteristics of the present invention, as well as methods of operation and functions of the related elements of the structure, and the combination of parts and economies of manufacture, will become more apparent upon consideration of this detailed description with reference to the figures which accompany this application.

### BRIEF DESCRIPTIONS OF THE DRAWINGS

The novel features which are believed to be characteristic of the present invention, and related systems and methods according to the present invention, as to their structure, organization, use and method of operation, together with further objectives and advantages thereof, may be better understood from figures which accompany this application, in which presently preferred embodiments of the invention are illustrated by way of example. It is expressly understood, however, that any such figures are for the purpose of illustration and description only, and not intended as a definition of the limits of the invention. In the accompanying figures:

FIG. 1 depicts the major components as well as sub-components of each major component of one embodiment of the present invention;

FIG. 2 is a block diagram of the fluidic components in accordance with one embodiment of the present invention;

FIG. 3 is a block diagram of the fluidic components with recirculation capabilities in accordance with one embodiment of the present invention;

FIG. 4A depicts the symbol for the fluidic resistor and fluidic capacitor in FIG. 4B, FIG. 5A, and FIG. 5B;

FIG. 4B is a fluidic resistance and capacitance equivalent circuit diagram in accordance with one embodiment of the present invention;

FIG. 5A and FIG. 5B combines to show a fluidic resistance and capacitance equivalent circuit diagram in accordance with a second embodiment of the present invention;

FIG. 6 is a front perspective view of the human lower extremity active compression apparel system in accordance with one embodiment of the present invention where knee joints are the addressed anatomical portion of the human body;

FIG. 7 is a front perspective view of the human lower extremity active compression apparel system in accordance with a second embodiment of the present invention where knee joints are the addressed anatomical portion of the human body;

FIG. 8 is a front perspective view of the human lower extremity active compression apparel system in accordance with a third embodiment of the present invention where knee joints are the addressed anatomical portion of the human body;

FIG. 9 is a lateral side perspective view of the active compression apparel system in accordance with an embodiment of the present invention where the left knee joint is the addressed anatomical portion of the human body;

FIG. 10 is a medial side perspective view of the active compression apparel system in accordance with the embodiment shown in FIG. 9;

FIG. 11 is an anterior perspective view of the active compression apparel system in accordance with the embodiment shown in FIG. 9;

FIG. 12 is a posterior perspective view of the active compression apparel system in accordance with the embodiment shown in FIG. 9;

FIG. 13A is an anterior perspective view of the active compression apparel system in accordance with another embodiment of the present invention where the left knee joint is the addressed anatomical portion of the human body;

FIG. 13B is a close-up view of the air microfluidic module integrated with garment of the active compression apparel system in accordance with the embodiment shown in FIG. 13A;

FIG. 14A is an anterior perspective view of the active compression apparel system in accordance with yet another embodiment of the present invention where the left knee joint is the addressed anatomical portion of the human body;

FIG. 14B is a close-up view of the air microfluidic module integrated with garment of the active compression apparel system in accordance with the embodiment shown in FIG. 14A;

FIG. 15 is an anterior perspective view of the active compression apparel system in accordance with an embodiment of the present invention where only the medial and medial anterior sides of the left knee joint is the addressed anatomical portion of the human body;

FIG. 16 is a diagrammatical cross-sectional view of the active compression apparel garment system in accordance with an embodiment of the present invention;

FIG. 17A is an anterior view of a two-piece external actuation garment system in accordance with an embodiment of the present invention where the right knee joint is the addressed anatomical portion of the human body;

FIG. 17B shows one piece of the external actuation garment as depicted by FIG. 17A;

FIG. 17C shows the second piece of the external actuation garment as depicted by FIG. 17A;

FIG. 17D shows the overlapping portions of the two garments as depicted by FIG. 17A;

FIG. 18 is an anterior perspective view of an external actuation garment in accordance with an embodiment of the present invention where the right knee joint is the addressed anatomical portion of the human body;

FIG. 19 is a posterior perspective view of an external actuation garment in accordance with an embodiment of the present invention where the left knee joint is the addressed anatomical portion of the human body;

FIG. 20 is a side view of an external actuation garment in accordance with an embodiment of the present invention where the right knee joint is the addressed anatomical portion of the human body;

FIG. 21 is an exploded perspective view of an air microfluidics channel network module in accordance with one embodiment of the present invention;

FIG. 22A is an isometric perspective view of the air microfluidics socket integrated with garment as seen in FIG. 21;

FIG. 22B is a side perspective view of the air microfluidics socket integrated with garment of the embodiment of FIG. 22A;

FIG. 22C is a cross-sectional view of the air microfluidics socket integrated with garment of the embodiment of FIG. 22B;

FIG. 22D is yet another cross-sectional view of the air microfluidics socket integrated with garment of the embodiment of FIG. 22B;

FIG. 23A is a side perspective view of the air microfluidics chip where the side is made transparent to see the internal channels as seen in FIG. 21;

FIG. 23B is a cross-sectional view of the air microfluidics chip of the embodiment of FIG. 23A;

FIG. 23C is yet another cross-sectional view of the air microfluidics chip of the embodiment of FIG. 23A;

FIG. 24 is an isometric perspective view of an air microfluidics channel network module in accordance with a second embodiment of the present invention;

FIG. 25A is an isometric perspective view of the air microfluidics chip integrated with garment where the walls are made transparent to show the internal channels as seen in FIG. 24;

FIG. 25B is a top perspective view of the air microfluidics chip integrated with garment where the walls are made transparent to show the internal channels of the embodiment of FIG. 25A;

FIG. 25C is a cross-sectional view of the air microfluidics chip of the embodiment of FIG. 25B;

FIG. 25D is another cross-sectional view of the air microfluidics chip of the embodiment of FIG. 25B;

FIG. 25E is yet another cross-sectional view of the air microfluidics chip of the embodiment of FIG. 25B;

FIG. 26A is an isometric perspective view of the elastic mini channel network fully integrated with garment as seen in FIG. 24;

FIG. 26B is a back perspective view of the elastic mini channel network fully integrated with garment of the embodiment of FIG. 26A;

FIG. 27 shows four different exemplary shapes of the balloon actuators;

FIG. 28 shows an embodiment of elastic mini channel network fully integrated with garment with two mini channels for fluidic transportation connecting to two balloon actuators;

FIG. 29 shows the size and scale of various embodiments of the hardware components of the invention described herein with comparison to the human lower extremity;

FIG. 30 depicts a flow chart of a series of events for the fluidic system of one embodiment of the present invention required for the inflation/pressurization of the balloon actuators;

FIG. 31 depicts a flow chart of a series of events for the fluidic system of one embodiment of the present invention required for the deflation/depressurization of the balloon actuators; and

FIG. 32 depicts a flow chart of a series of events for the sensor systems and control system of one embodiment of the present invention.

It is to be understood that the accompanying drawings are used for illustrating the principles of the embodiments and exemplifications of the invention discussed below. Hence the drawings are illustrated for simplicity and clarity, and not necessarily drawn to scale and are not intended to be limiting in scope. Reference characters/numbers are used to depict the elements of the invention discussed that are also shown in the drawings. The same corresponding reference number is given to a corresponding component or components of the same or similar nature, which may be depicted in multiple drawings for clarity. Text may also be included in the drawings to further clarify certain principles or elements of the invention. It should be noted that features depicted by one drawing may be used in conjunction with or within other drawings or substitute features of other drawings. It should further be noted that common and well-understood elements for creating a commercially viable version of the embodiments of the invention discussed below are often not depicted to facilitate a better view of the principles and elements of the invention discussed below

### DETAILED DESCRIPTION OF PREFERRED EMBODIMENTS

In the following discussion, the accompanying figures pertain to the preferred embodiments, and the description is not intended to limit the scope, applicability or configuration of the invention as described by the claims. The description enclosed herein aims to provide any person skilled in the art the necessary information for the implementation of the preferred embodiments of the invention described herein.

Below is some clarification for certain terminologies; it must be noted that the clarifications do not limit the scope of the meaning of the terminologies in the context of the relevant art, and the invention described herein.

“Anatomical portion” comprises the meaning of any part of the human body including but not limited to body joints and limbs.

“Pathways” comprises the meaning of any component that transports a fluidic or electrical current or both including but not limited to tubing, channels, wiring, and traces.

“Fluidic resistance algorithm” comprises the meaning of any principle that can affect the flow rate and pressure of a fluid.

“Mini channel” have cross-sectional characteristic lengths from and including 3 mm down to 200 μm; “micro channel” have cross-sectional characteristic lengths from and including 200 μm down to 1 μm. It must be noted that the fluidic channel classification scheme is arbitrary and is used for clarity while not limiting the scope of any embodiments of the invention disclosed herein.

“Balloon” comprises the meaning of any device of any size that can inflate and deflate via fluidic pressurization and depressurization.

Although air is used to describe the principle, operation and function of the invention described herein, any fluid can be used with or replace air to achieve the desired goal of the invention described herein.

Miniaturized components including but not limited to mini air pumps, mini valves, mini tubing and mini channels may be used with or replaced by even smaller components on the microscale for certain embodiments of the invention described herein.

Additionally, singular forms including but not limited to “a” and “an”, may also comprise the meaning of plural forms as well, unless explicitly stated otherwise.

### i. System Overview

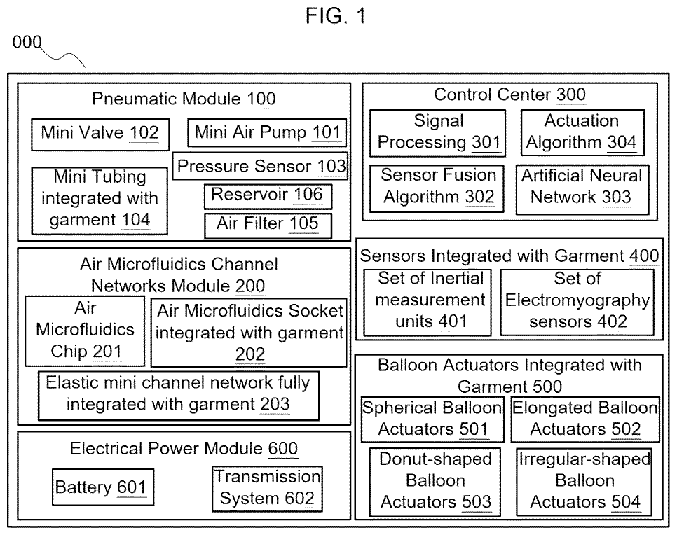

Air microfluidics and air minifluidics enabled active compression apparel involves many hardware components and software components. It differentiates from the traditional pneumatic system and pneumatic microfluidics logic circuit by its scale, system integration, implementation, components, control, operational principles and fabrications. FIG. 1 shows the major modules that make up the overall system 000 as well as the subcomponents that make up each module. The overall system 000 consists of a pneumatic module 100, an air microfluidics channel networks module 200, a control center 300, balloon actuators integrated with garment 500, sensors integrated with garment 400, and an electrical power module 600. Various embodiments and exemplifications of the present invention are not limited by the modules mentioned above; additional modules can be added to the system 000 to produce commercially-viable versions of the invention described herein.

The pneumatic module 100 is mainly used to generate the airflow and air pressure by using miniature versions of traditional industrial or macro-sized pneumatic components; the pneumatic module 100 comprises mini air pumps 101, mini valves 102, pressure sensors 103, mini tubing integrated with garment 104, fluidic reservoirs 106, and air filters 105.

The air microfluidics channel networks module 200 is the most important aspect of the present invention which induces fluidic pressurization/depressurization delays from the principle of equivalent hydraulic resistance and acts as an airflow selection manifold; the air microfluidics channel networks module 200 comprises of at least one air microfluidics chip 201, at least one air microfluidics socket integrated with garment 202, and at least one elastic mini channel network fully integrated with garment 203. The importance of the air microfluidics channel networks module 200 cannot be understated; it allows for robust, reliable and mechanically simple, yet functionally complex actuation of the balloon actuators, without adding any bulk to either the hardware system or the balloon actuators. Hence, it allows for ultra formfitting, ultra lightweight, and aesthetically pleasing active compression apparel, and more generally soft wearable robots. Furthermore, it can be hot water washed with detergent, and heat tumble dried using conventional washing and drying machines. For certain embodiments of the invention described herein, modules and components including but not limited to air microfluidic chip 201, pneumatic module 100, control center 300, and electrical power module 600, may be detached from garments to facilitate cleaning of the garment and prolonging the life of the components of the system 000. Fabrication wise, air microfluidics channel networks module 200 may be customized and/or mass produced using any of the traditional plastic moulding techniques, any of the 3D printing techniques, via softlithrography, or any reduction and addition manufacturing procedures or combinations thereof. Integration of the air microfluidics channel network module 200 with garment can be achieved through any appropriate textile lamination techniques and sewing techniques.

The control center 300 comprises signal processing components 301, a sensor fusion algorithm 302, artificial neural network 303, and actuation algorithm 304. The control center 300 may also comprise any common and well-understood elements that would be necessary to produce a commercially viable control center 300 for the system 000; these elements include but not limited to a motherboard, central processing unit (CPU), data storage in the form of solid state drives (SSD), wireless network systems, random access memory (RAM), various electrical subcomponents such as electrical resistors, capacitors, diodes, fuses, and various electronic subcomponents such as field-effective transistors and any other types of silicon transistors.

The sensors integrated with garment 400 provides the signals of information including but not limited to the user's biometric and kinematics for the control center to tailor the active compression to an anatomical portion of the user's body; the sensors integrated with garment 400 includes but not limited to a set of inertial measurement units (IMU) 401 and a set of electromyography sensors (EMG) 402. The IMU sensors 401 may comprise a combination of accelerometers, gyroscopes, and magnetometers. The IMU sensors 401 can be of various degrees of freedom. The EMG sensors 402 can be of any type that uses electrodes of any type. The number, location, and type of sensors integrated with garment 400 depend on the application, the embodiment of the current invention, and the anatomical portion of the human body where the active compression is applied to or used for.

The balloon actuators integrated with garment 500 is another key component. It differentiates from other soft actuators within the family of soft fluidic actuators often used in soft wearable robots and haptic devices and other soft pneumatic wearable actuators such as but not limited to McKibben pneumatic artificial muscles regarding but not limited to implementation, functionality, materials and fabrication. The pneumatic balloon actuators integrated with garment 500 comprise a combination of at least one of spherical balloon actuators 501, elongated balloon actuators 502, donut-shaped balloon actuators 503, or irregular-shaped balloon actuators 504. It must be noted that the shapes described above are the preferred shapes, but balloon actuators integrated with garment 500 of any shape and size is within the scope of the invention described herein. Similar to the sensors integrated garment 400, the number, location, and type of balloon actuators integrated with garment 500 depends on the application, embodiment of the invention described herein, and the anatomical portion of the human body where the active compression is applied to or used for.

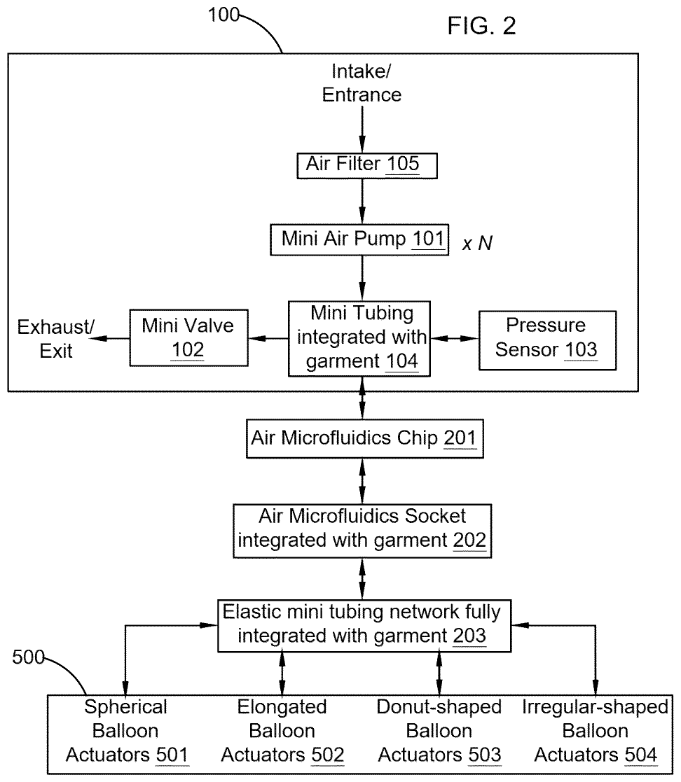

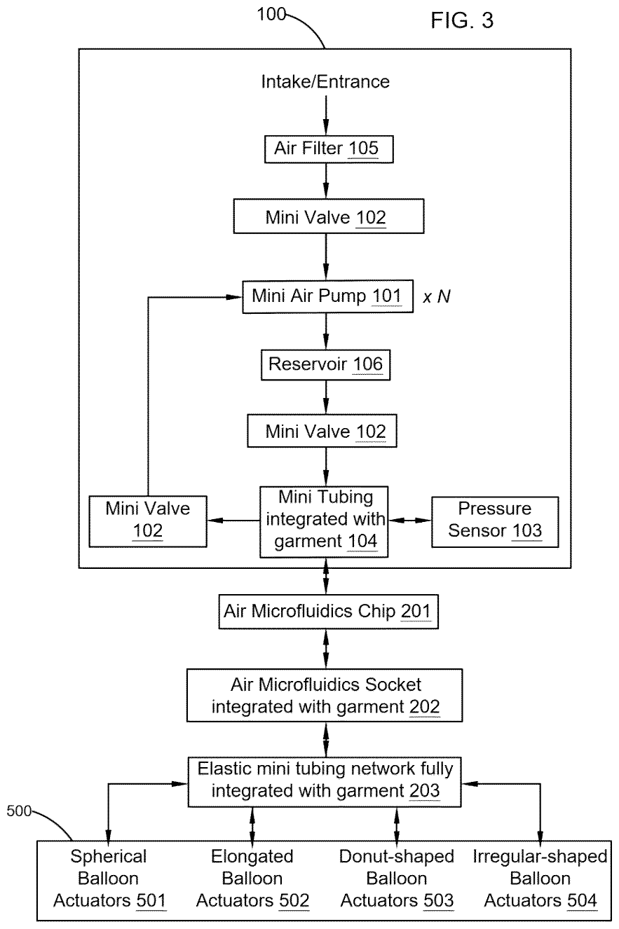

Looking at just the flow and transport of air for one embodiment of the invention described herein, where there is no recirculation of air, of the present invention as shown by FIG. 2. at least one mini air pump 101 draws the air from the external/ambient environment through at least one replaceable air filter 105 which keeps out unwanted particles, elements and moisture from the internal components. The bidirectional arrows shown in FIG. 2 as well as FIG. 3 represents air pathways that allow air flow in both the inflation/pressurization direction towards the balloon actuators integrated with garment 500 as well as the deflation/depressurization direction towards exhaust/exit of the pneumatic module 100. The single direction arrows shown in FIG. 2 and FIG. 3 represents the air pathways that allows only single directional flows. It also must be noted that the physical embodiments of the arrows shown in FIG. 2 and FIG. 3 are air pathways which include but not limited to tubing, mini tubing, air microfluidic channels, and air minifluidic channels. The air pathways can be fully integrated, partially integrated, or not at all integrated with the garment. The at least one mini air pump 101 then delivers the air into at least one mini tubing integrated with garment 104 downstream, which is connected in parallel to at least one pressure sensor 103 that measures the air pressure within the internal fluidic pathways of the pneumatic module 100. It must be noted that for certain embodiments of the invention described herein, the at least one pressure sensor 103 may measure the air pressure of the entire system or part of the system or both. The at least one mini tubing integrated with garment 104 is connected for two direction flow with a microfluidics chip 201 and at least one mini valve 102 for exhausting the air flow and air pressure into the external/ambient atmosphere. The mini valve 102 can be either normally closed or normally open valves of any type with any number of ports and positions, or for the preferred embodiment, a normally closed 2 port 2 position mini solenoid valve; this will apply to all mentions of 102 in this patent unless otherwise explicitly stated. When the mini valve 102 is open, the air is exhausting into ambient atmosphere, otherwise, the air flows into the air microfluidics chip 201, which may be mechanically or magnetically attached to the air microfluidics socket integrated with garment 202, then through the elastic mini tubing network fully integrated with garment 203 and into various balloon actuators 501, 502, 503, 504. In certain embodiments of the invention described herein, at least one air microfluidics chip 201 may be fully, or partially, or not at all integrated with a garment with or without the need of having to be attached to at least one air microfluidics socket integrated with garment 202.

In another embodiment of the present invention, recirculation is incorporated into the pneumatic module, as seen in FIG. 3. The differences between the recirculation embodiment (FIG. 3) and the non-recirculation embodiment (FIG. 2) are in the pneumatic module 100. At least one mini valve 102 connects at least one mini air pump and the at least one air filter 105 to draw air from the ambient/external atmosphere, and the said mini valve(s) permits the mini air pump(s) to draw air from the external/ambient atmosphere when necessary. Downstream from the mini air pump(s) is at least one fluidic reservoir 106 which is a container that can be made out of any material and of any shape that can hold up to and beyond the maximum pressure at which the mini air pump(s) is still able to produce a flow rate, this pressure is denoted as the maximum backpressure of the system. Multiple fluidic reservoirs 106 may be connected in parallel to increase the volume of the reserved air. The purpose of the fluidic reservoir 106 is to increase efficiency and improve the response of the system through the use of stored compressed air. At least one mini valve 102 connects the at least one fluidic reservoir 106 and the at least one mini tubing integrated with garment 104 downstream; the function of this said mini valve(s) 102 is to allow for recharge of the fluidic reservoir(s) 106. Instead of exhausting the air during deflation/depressurization of the balloon actuators integrated with garment 500 into external/ambient atmosphere, at least one mini valve 102 allow recirculation of the air from the mini tubing integrated with garment 104 by connecting with mini air pump(s) 101 which actively draws air into the fluidic reservoir(s) 106 completing the recirculation air pathway.

### ii. Principle and Implementation of Equivalent Hydraulic Resistance

It is well known that air is a compressible fluid; in the strictest sense, compressible fluid means that the density of the said fluid changes with changing volume given the same mass of the said fluid. The opposite of compressible fluid is, of course, incompressible fluid, which has constant density under all conditions. However, there is no true incompressible fluid, and even a liquid is slightly compressible under high pressure. Hence, an assumption can be made that any fluid that does not change too much in density during laminar flow with Mach number less than 0.3 can be considered an incompressible fluid. The flow and pressure change for air microfluidics and air minifluidics of the present invention precisely satisfies the above criteria to validate the assumption of air as an incompressible fluid. It must be noted that there would be errors with the assumption that the air is incompressible for this invention. However, the errors are tolerable and the equivalent hydraulic resistance is used as a design principle and engineering estimation instead of fundamental theory. Hence, the air microfluidics system and air minifluidics system can be modelled using equivalent hydraulic resistance and electrical circuit analogy. The reason that it is named equivalent hydraulic resistance because it is not a physical property of air, but a design parameter for air microfluidics and air minifluidics.

Hydraulic resistance denoted as R_{hyd} with the units

$$[ \frac{Pa \cdot s}{{m}^{3}} ] = [ \frac{k g}{{m}^{4} \cdot s} ] ,$$

relates to pressure drop denoted as Δp with the units

$$[ Pa ] = [ \frac{N}{{m}^{2}} ] = [ \frac{kg}{m \cdot {s}^{2}} ] ,$$

and volume flow rate denoted as Q with the unit

$$[ \frac{{m}^{3}}{s} ]$$

through Hagen-Poiseuille law: Δp=R_{hyd}·Q, which is completely analogous to Ohm's law, hence the naming of electrical circuit analogy. When combined with the Darcy-Weisbach equation,

$$\frac{Δ p}{L} = \frac{2 \cdot {f}_{F} \cdot {G}^{2}}{ρ \cdot {D}_{h}}$$

with the Fanning friction factor, denoted as f_{F}, a dimensionless number, equivalent hydraulic resistance can be determined. Within the Darcy-Weisbach equation,

$$\frac{Δ p}{L}$$

is the frictional pressure gradient with the unit

$$[ \frac{Pa}{m} ] ,$$

G is the mass flux with the unit

$$[ \frac{kg}{{m}^{2} \cdot s} ] ,$$

ρ is the density of the fluid with the unit

$$[ \frac{kg}{{m}^{3}} ] ,$$

and Dₕ is the equivalent hydraulic diameter with unit [m], which is simply a characteristic cross-sectional length of fluidic channels. Fanning friction factor is related to Reynold's number denoted as Re by f_{F}·Re=C, where C is a dimensionless empirical constant for various fluidic channel cross-sectional shape. Reynold's number is a dimensionless ratio of inertial forces and viscous forces with the following equation:

$$R e = \frac{G \cdot {D}_{h}}{µ} ,$$

where μ is the fluid's dynamic viscosity with the units

$$[ Pa \cdot s ] = [ \frac{kg}{m \cdot s} ] .$$

Further rearranging and substitution of the above equations, the equivalent hydraulic resistance of various fluidic channel cross-sectional shape and length can be determined, Henrik Bruus has consolidated a list of hydraulic resistance formulas for commonly used fluidic channel cross-sectional shapes in his book Theoretical Microfluidics (ISBN: 978-0-19-9233508-7).

Various embodiments of the invention described herein can use air microfluidic channels and air minifluidic channels of any cross-sectional shape, size, geometry, length and route. The preferred cross-sectional shapes of air microfluidics and air minifluidics channels are rectangles, circles, and squares.

The equations for the equivalent hydraulic resistance of the three preferred cross-sectional shapes of air microfluidic channels and air minifluidic channels are the following:

Circle:

$$\frac{8}{π} \cdot µ \cdot L \cdot \frac{1}{{a}^{4}} \qquad Eq . 1$$

Rectangle:

$$\frac{12 \cdot µ \cdot L}{1 - 0.63 \cdot ( \frac{h}{w} )} \cdot \frac{1}{{h}^{3} \cdot w} \qquad Eq . 2$$

Square:

$$28.4 \cdot µ \cdot L \cdot \frac{1}{{w}^{4}} \qquad Eq . 3$$

Where a is the radius of the circle for Eq. 1; h and w are respectively height and width of the rectangle for Eq. 2; w is the side length of the square for Eq. 3; μ and L are fluid dynamic viscosity and length of the fluidic channel for Eq. 1, Eq. 2, and Eq. 3. a, w, h and L all have the unit [m], where μ has the units

$$[ Pa \cdot s ] = [ \frac{kg}{m \cdot s} ] .$$

Similarly, equivalent hydraulic capacitance denoted C_{hyd} can be determined to be

$${C}_{h y d} = - \frac{d V}{d p} ,$$

where dV is change in fluidic volume and dp is change in fluidic pressure. Hydraulic capacitance is due to the compliant nature of elastomers and soft walls and enclosures. For instance, a compliant bladder needs to allow the volume to be fully occupied by fluid first before pressure increases. Since Hagen-Poiseuille law and Ohm's law are analogous, electric circuit theory, including Kirchhoff's law, paths of least resistant for parallel pathways among others can be applied to air microfluidics and air minifluidics. Again, it must be stressed that there would be errors in this analogy, as it is only completely accurate as Reynold's number approaches zero, and for long narrow channels far apart, but it is considered to be acceptable for design principles and used as an engineering tool for air microfluidics and air minifluidics. The resistances of air microfluidic and air minifluidic channels add in series, (i.e. Rₜₒₜₐₗ=R₁+R₂), and the additive law for the resistances of air microfluidic and air minifluidic channels arranged in parallel is the following: Rₜₒₜₐₗ⁻¹=R₁⁻¹+R₂⁻¹.

For all embodiments of the invention described herein, the paths of the least resistant analogy allow for a sequential delay in pressurization of at least one balloon actuator integrated with garment induced by parallel air microfluidic and air minifluidic channels. In other words, the balloon actuator(s) connected to the fluidic pathway of the least resistant will be pressurized first, then the balloon actuator(s) connected to the fluidic pathway of the second least resistant will be pressurized and so on. The reason that the paths of the least resistance analogy works very well with air microfluidics and air minifluidics is due to the fact that the characteristic lengths (i.e. equivalent hydraulic diameter) of the equivalent hydraulic resistance equations (Eq. 1, Eq. 2, Eq. 3) for the air microfluidic and air minifluidic channels are to the power of four. Hence, the cross-sectional size of the air microfluidic and air minifluidic channels induces the largest change in equivalent hydraulic resistance compared to other parameters in the said equations. Hence, as the fluidic channel cross-sectional size decreases, the equivalent hydraulic resistance increases exponentially.

For any embodiments of the invention described herein, fluidic channel classification scheme by Kandlikar and Grande (Heat Transfer Engineering 24(1):3-17 (2003)) will be employed and is described as follows. “Conventional channels” have cross-sectional characteristic lengths greater than 3 mm; “mini channels” have cross-sectional characteristic lengths from and including 3 mm down to 200 μm; “micro channels” have cross-sectional characteristic lengths from and including 200 μm down to 1 μm. It must be noted that the fluidic channel classification scheme is arbitrary and is used for clarity while not limiting the scope of any embodiments of the invention disclosed herein.

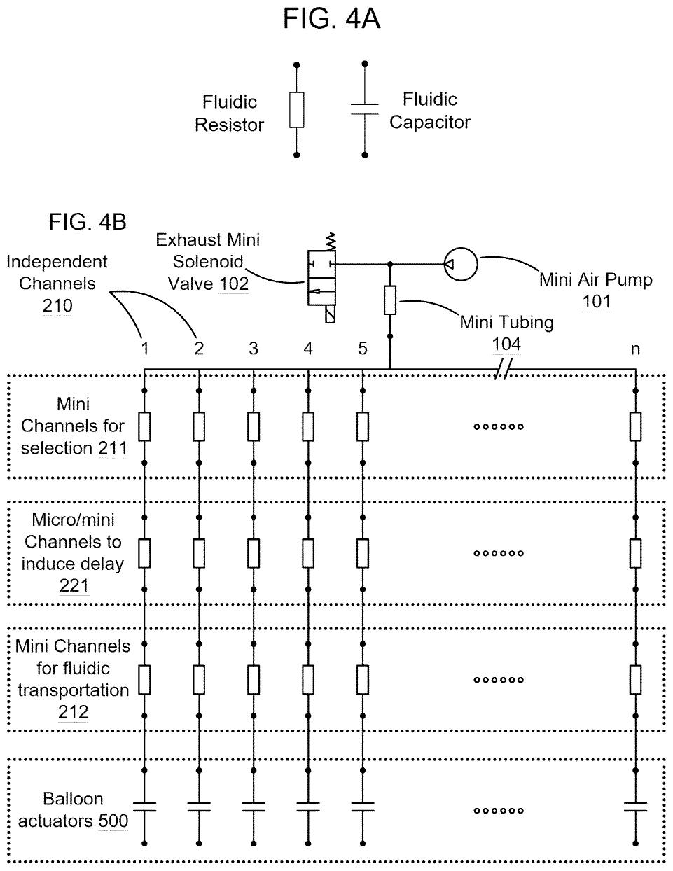

FIG. 4A shows the schematic symbol for fluidic resistors and fluidic capacitors. FIG. 4B is an equivalent hydraulic circuit schematic view of the fluidic system for one embodiment of the present invention. Please note that not all fluidic components are included in FIG. 4B for clarity. At least one mini air pump 101 produces the pressure and flow within the fluidic system. The mini tubing 104, which has small equivalent hydraulic resistance compared to the air microfluidic and air minifluidic channels causes practically no pressure drop in transporting air; hence it could be much longer than the rest of the fluidic pathways allowing for placing the pneumatic module container in a preferred location on the human body. The mini tubing 104 feeds multiple independent channels 210 that are arranged in a parallel circuit configuration to induce sequential delays in the pressurization of the balloon actuators integrated with garment 500.

The number of independent parallel channels 210 that can be implemented in any embodiments of the invention described herein are determined by the applications and the anatomical portions of the human body where the active compression apparel is addressing. The mini channels for selection 211 may be part of the air microfluidics chip. As mentioned earlier, in certain embodiments of the present invention, the air microfluidics chip may be permanently integrated with a garment; hence, the mini channels for selection 211 do not exist. However, for certain embodiments of the present invention, where the air microfluidics chip is detachable from the air microfluidics socket integrated with garment, then the mini channels for selection 211 allows for a modular system, which the user can choose which balloon actuators 500 to inflate and which not to inflate by selecting and/or changing the air microfluidics chip.

Regarding equivalent hydraulic resistance, similar to previously mentioned mini tubing 104, mini channels for selection 211 has a very low equivalent hydraulic resistance compared to micro/mini channels to induce delay 221. The micro/mini channels to induce delay 221 have various channel cross-sectional characteristic length ranging from but not limited to 2 mm to 1 μm. Mini channels for fluidic transportation 212 are fluidic pathways for air to flow into the balloon actuators without introducing any significant equivalent hydraulic resistance. For each independent channels 210, the fluidic resistance of each element is added in series, and the compliance or fluidic capacitance of each element can be neglected as their values are negligible compared to the fluidic capacitance of the balloon actuators 500. The balloon actuators 500 are modeled as fluidic capacitance due to the compliant nature of the material making up its walls. These materials include but not limited to any polymers or elastomers or both.

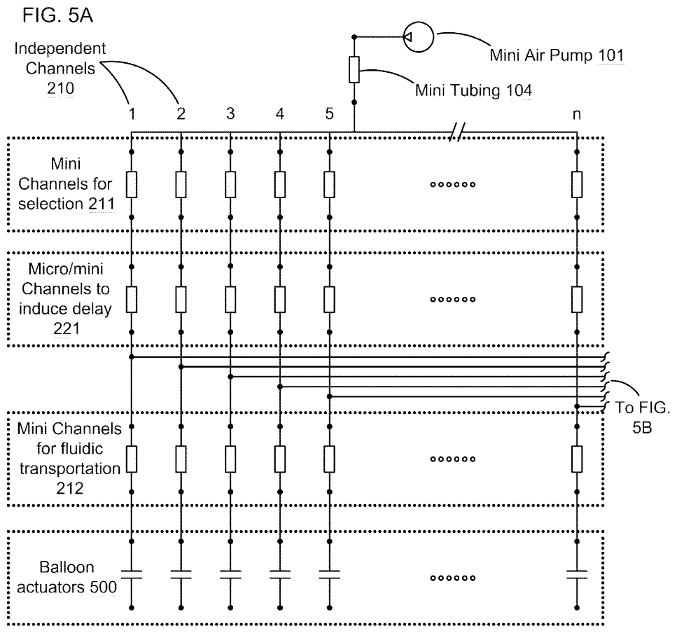

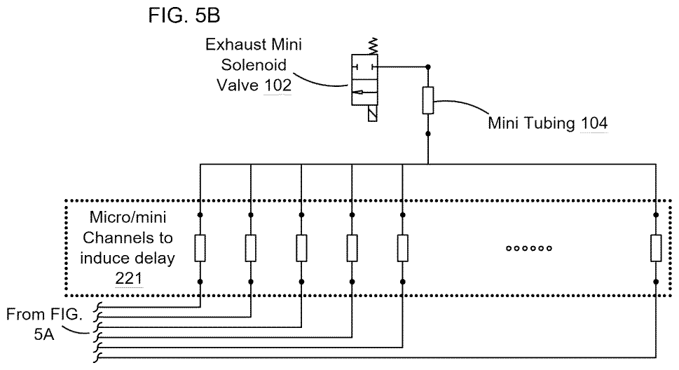

FIG. 5 is made up of two sub schematic drawings, FIG. 5A and FIG. 5B are equivalent hydraulic circuit schematic view of the fluidic system for an embodiment of the invention described herein. The difference between this embodiment and another embodiment depicted by FIG. 4B and described above is that the inflation/pressurization fluidic pathway is different than the deflation/depressurization fluidic pathway. Therefore, the inflation sequence and deflation sequence can be separately programmed by different micro/mini channels to induce delay 221.

### iii. Active Compression Apparel with Example for the Knee Joint

As mentioned earlier, many components including but not limited to garment and electronics are not drawn in the figures to better show the principles of operations and for clarity of showing the components of the system(s) intended to be described by each drawing. Each drawing disclosed in this section (3. Active Compression Apparel with Example for the Knee Joint) can supplement for each other as well as drawings from any other sections.

Air microfluidics and air minifluidics enable the creation of various active compression apparel. This section shows various embodiments of the active compression apparel for the knee joint; this, however, does not limit the scope of the invention disclosed herein, rather for disclosing a person skilled in the art the principles of designing air microfluidics and air minifluidics enabled active compression apparel for any anatomical portion(s) of the human body. Furthermore, any modifications and changes may be made to the embodiments shown herein without departing from the scope of the invention disclosed herein. It must be noted that all the drawings described in this section are two-dimensional sketches/drawings depicting three-dimensional features, objects, and surfaces. Therefore, certain elements of the drawings are not to scale and are only intended to show various aspects of the invention disclosed herein so a person skilled in the art can faithfully recreate any embodiments of the invention disclosed herein.

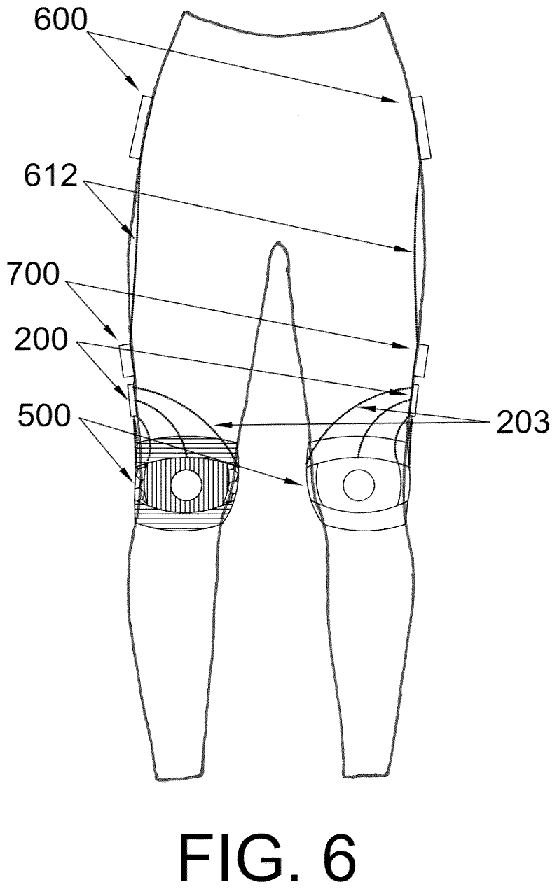

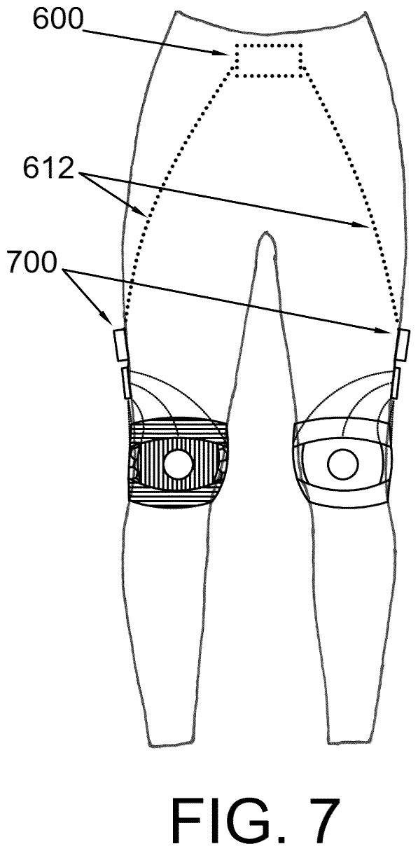

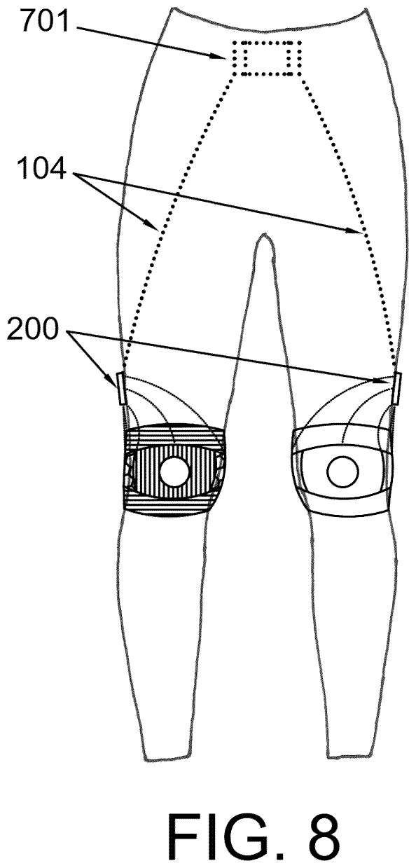

FIG. 6, FIG. 7, and FIG. 8 show three different placements of various modules on the human body as viewed from the front, specifically the lower extremity (i.e. leg) for three different embodiments. The embodiment shown in FIG. 6 illustrates a system where each leg has independent hardware systems. The at least one electrical power module 600 is located around or on the lateral/outside surface of the upper thigh, one for each leg. The pneumatic module and the control center for each leg is preferably enclosed in the same container 700 and is located around or on the lateral/outside surface of the lower thigh, and they are connected to the electrical power module 600 by at least one electrical pathway 612 including but not limited to electrical wiring, electrical traces and conductive fabric. The air microfluidics channel network modules 200 are located beneath the pneumatic module and control center containers 700. The balloon actuators integrated with garment 500 is surrounding the knee joint, above, below, inside, outside, front and back. Elastic mini channel network fully integrated with garment 203 connects the air microfluidics channel network module 200 and balloon actuators 500. The left and right knee joints may have different sets of balloon actuators integrated with garment 500. Furthermore, different zones around the knee joint can have different sets of balloon actuators integrated with garment 500. For instance, the top portion of the knee, around the quadriceps tendon may have different numbers of and differently shaped balloon actuators 500 than the bottom portion of the knee surrounding the patellar tendon. In more general terms, the electrical power module 600 is the most proximally located to the body's center of mass, the pneumatic module and the control center container 700 is more distally located above the knee joint, and the air microfluidics channel network module 200 is the most distally located just above the knee joint.

In another embodiment of the invention described herein, a single electrical power module 600 is preferably located behind the user at the waist near the tail bone region as seen in FIG. 7. The dotted line box represents the electrical power module 600 on the back of the hip or waist. Depending on the number of anatomical portions of the human body the balloon actuators are addressing, at least an equivalent number of electrical pathways 612 connects the single electrical power module 600 with the at least same number of pneumatic module and control center containers 700. The rest of the embodiment shown in FIG. 7 has the same configuration as the embodiment shown in FIG. 6. In yet another embodiment of the invention described herein, the at least one electrical power module, the at least one pneumatic module and the at least one control center are enclosed in a single container 701 addressing all the balloon actuators in all the anatomical portions for one apparel or multiple apparels as shown in FIG. 8. The location of this single container 701 is preferably located near the human body's center of mass; in other words, the said container 701 is more proximally located than the other components. Furthermore, instead of electrical pathways exiting this single container, at least one mini tubing integrated with garment 104 or fluidic pathway connects it with at least one air microfluidics channel networks module 200. The rest of the embodiment shown in FIG. 8 has the same configuration as the embodiment shown in FIG. 6.

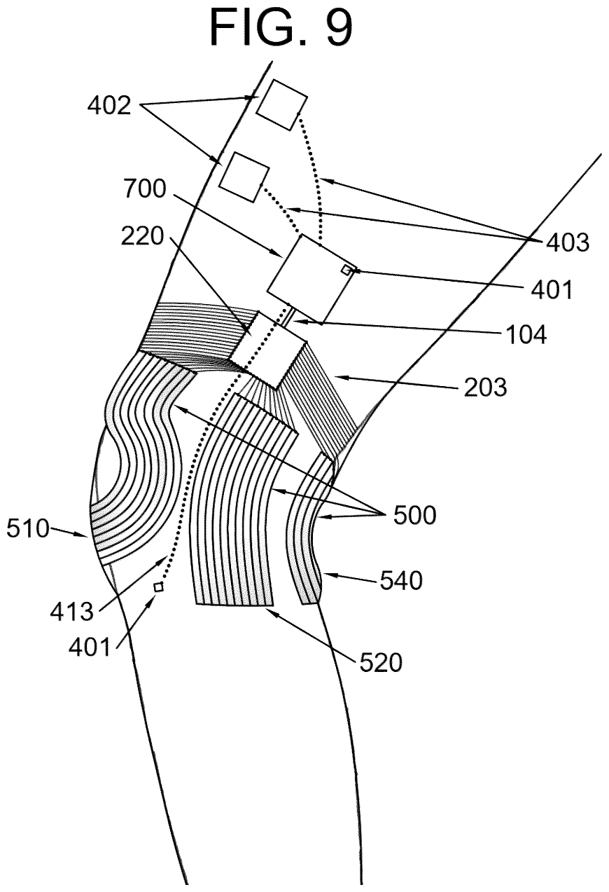

FIG. 9 shows the side view of one embodiment of the invention described herein. The system depicted by FIG. 9 is an active compression apparel for the human knee joint enabled by air microfluidics and air minifluidics. The knee is viewed from the outside/lateral side of the left knee joint. The purpose of this system is for minimizing the risks of injury of a healthy human knee joint as well as reduce pain and increase the physical function of an injured knee joint through active compression. The active compression increases joint stability, joint proprioception, skin temperature, and bodily internal fluid flow while decreasing the joint load. The pneumatic module and the control center are enclosed in a single container 700, which may be fully integrated with the garment or made to be detachable from the garment. The air microfluidics channel networks module container 220 which contains air microfluidics chip and air microfluidics socket integrated with garment is directly below or more distally located than the pneumatic module and control center container 700. The two containers 220, 700 are connected by at least one mini tubing integrated with garment 104 or at least one fluidic pathway. Elastic mini channel network fully integrated with garment 203 connects each balloon actuator integrated with garment 500 with the air microfluidics channel networks module container 220. The depicted balloon actuators integrated with garment 500 are elongated balloon actuators and irregular-shaped balloon actuators; however, any type of balloon actuators integrated with garment 500 may be used in any embodiments of the invention described herein. The balloon actuators integrated with garment 500 are separated into four sections in this embodiment of the invention described herein, and three of which are visible from the view in FIG. 9. The anterior balloon actuators 510 are addressing the patella, patellar tendon, quadriceps tendon and surrounding soft tissues. The lateral/outside balloon actuators 520 address the lateral soft tissues including but not limited to the iliotibial band, lateral collateral ligament, lateral meniscus and hamstring tendon. The posterior balloon actuators 540 address various posterior soft tissues as well as general knee stability and knee loading. Two IMU sensors 401, one of which is located inside the container 700 for the pneumatic module and control center located on the thigh; the other IMU sensor 401 is located on the shank portion of the apparel. At least one electrical pathway, preferably in the form of wire or trace embedded within the garment 413 connects the said IMU sensor 401 with the control center. The electrical pathway 413 supplies electrical power to the IMU sensor 401 from the control center and also sends signals from the IMU sensor 401 to the control center. The IMU sensors 401 consist of any combinations of accelerometers for measuring changes in linear acceleration of the object attached to the IMU sensor 401, gyroscopes for measuring changes in angular velocity of the object attached to the IMU sensor 401, and magnetometer for measuring the magnetic field around the object attached to the IMU sensor 401. Through software within the control center or software on a portable computing device, the signals from the IMU sensors 401 can be processed into a complete set of three dimensional kinematics motion data of the anatomical portion where the IMU sensors 401 are attached to including but not limited to angular position, angular velocity, angular acceleration, linear position, linear velocity, and linear acceleration. Furthermore, the IMU sensors 401 may have any number of axis, preferable 9 axes with all three subcomponents, 3-axis accelerometer, 3-axis gyroscope, and 3-axis magnetometer for measuring the most accurate and precise data. At least two IMU sensors 401 are required in the embodiment shown in FIG. 9 due to the fact that the anatomical portion is the knee joint which connects the thigh to the shank. The knee joint segment angular kinematic information can be determined through taking the differences of the absolute global angles between the two IMU sensors 401 attached to the thigh and the shank. Any number of IMU sensor 401 may be used for any embodiments of the invention described herein addressing any anatomical portions of the human body.

FIG. 9 further shows that at least one surface EMG sensor 402 is used to provide biometric signals of the muscles groups surrounding the knee joint in conjunction with the IMU sensors 401. The EMG sensor electrodes 402 is preferably located on rectus femoris; however, any quadriceps muscle group can provide the EMG signal. Two EMG sensor electrodes 402 are used in this embodiment of the invention described herein. The EMG sensor electrodes 402 can be any type including but not limited to conductive gel, metal, conductive foam-pad, and conductive fabric. The EMG sensor picks up the bioelectrical signal from muscle activity, which can then be interpreted through software to convey the amount of force imparted on the limbs and/or joints by a muscle group. In certain static posture and movement patterns, the EMG sensors 402 supplements the IMU sensors 401 with determining the movement and activity of the anatomical portion. For example, an IMU sensor is insufficient at distinguishing a person standing still with no significant muscle exertion versus when the same said person is standing while fully extending the leg to reach up; in both scenarios, the IMU sensor 401 has an identical signal output of zero knee segment angle change; however, the EMG sensor 402 can pick up the isometric muscle contractions of the quadriceps muscle groups, hence allowing for tailored compression of the knee joint via balloon actuators 500. It must be noted that certain embodiments of the invention described herein may not need to have both EMG sensors 402 and IMU sensor 401, and may have different types of biometric sensor altogether.

FIG. 10 shows the same embodiment of the invention described herein as FIG. 9, but from the inside/medial view of the left knee joint instead of the outside/lateral view of the left knee joint. In this view, the medial/inside balloon actuators 530 is visible. The medial/inside balloon actuators 530 are addressing medial soft tissues of the knee joint including but not limited to the medial meniscus, mediopatellar plica, vastus medialis and medial collateral ligament. The anterior balloon actuators 510 and posterior balloon actuators 540 are also visible. The elastic mini channel network fully integrated with garment 203 wraps around the front and the back of the knee joint connecting the balloon actuators with the single air microfluidics channel networks module container 220 on the lateral/outside surface (i.e. thigh) just above the left knee joint as seen in FIG. 9.

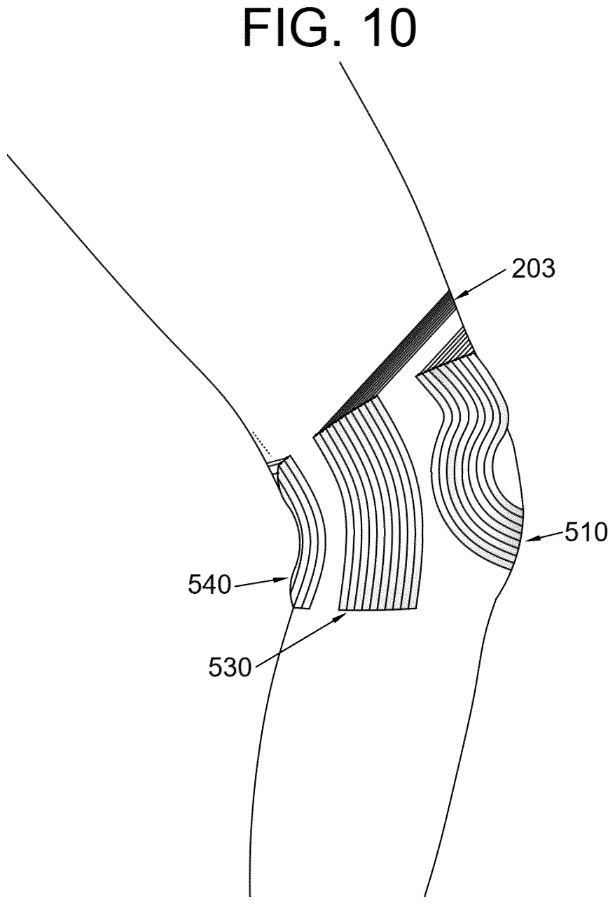

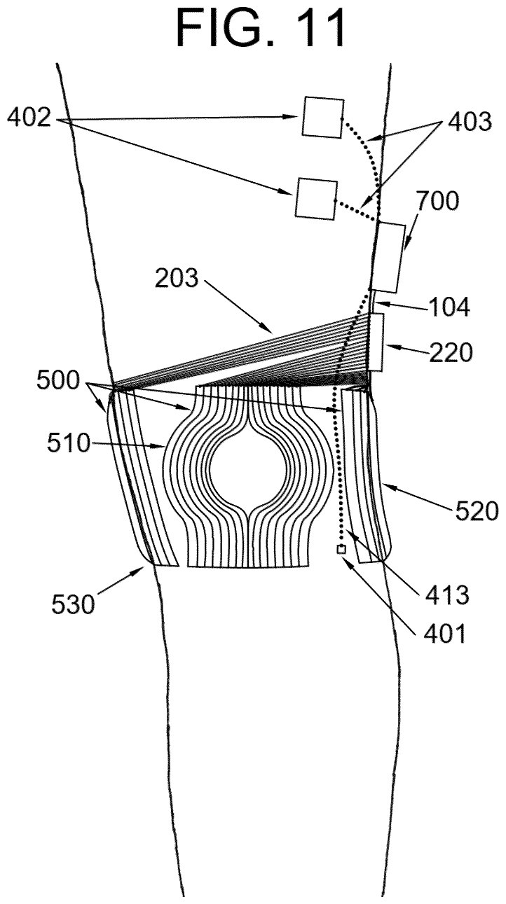

FIG. 11 and FIG. 12 each respectively shows the front/anterior view and the back/posterior view of the same embodiment of the invention described herein as FIG. 9 and FIG. 10. The anterior balloon actuators 510 surround the patella and hugs the sides of it without overlapping the top of the patella as seen in the front view. The reason for this implementation is not to apply direct compressional pressure pushing the patella into the patellar groove, which is unbeneficial but instead supports the patella by applying compressional pressure from the sides, which is beneficial. In the back/posterior view (FIG. 12), balloon actuators 500 that are larger in size compared to the other balloon actuators on the front and sides of this embodiment are shown. Reasons for the larger balloon actuators 500 on the back of the knee joint have to do with the curvature of the knee joint as well as the functionality of the balloon actuators 500 addressing the back/posterior of the knee. The front/anterior view FIG. 11 and the back/posterior view FIG. 12 also show the preferred thickness of the air microfluidics channel networks module container 220 and the container 700 for the pneumatic module and the control center compared to the size of the human leg. Preferably, the thickness of both containers 220, 700 are less or equal to 10 millimeters.

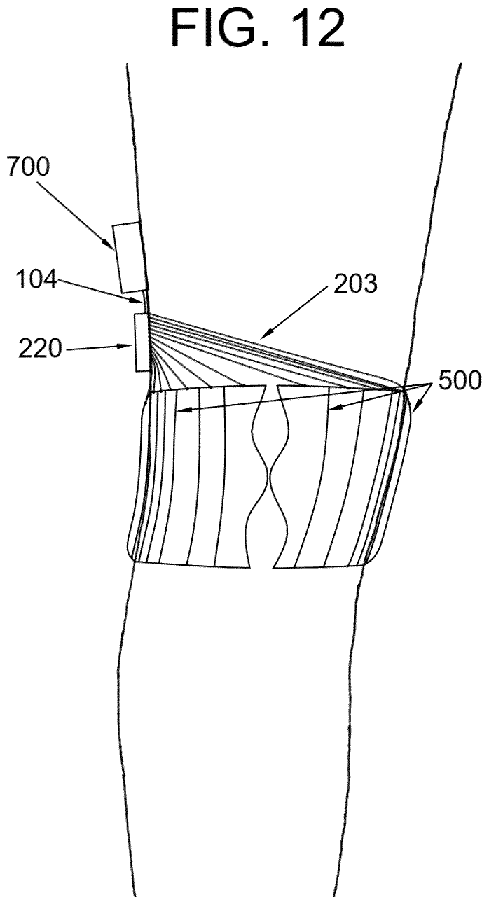

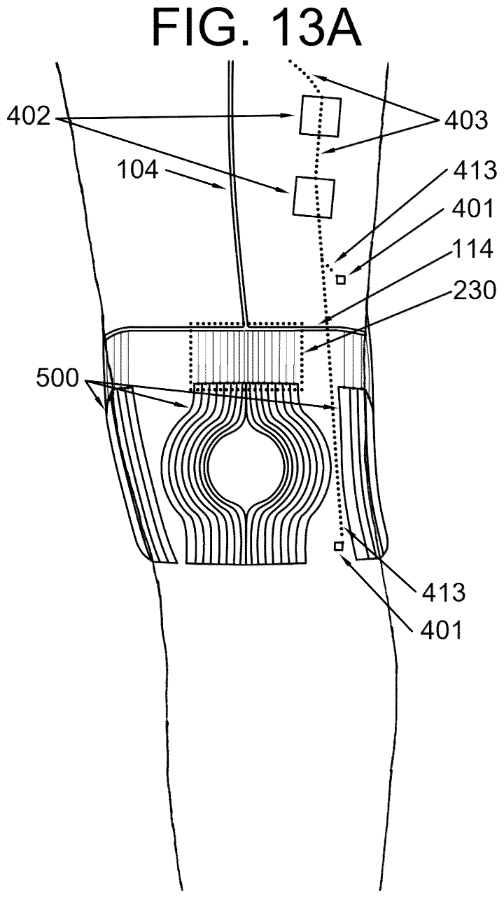

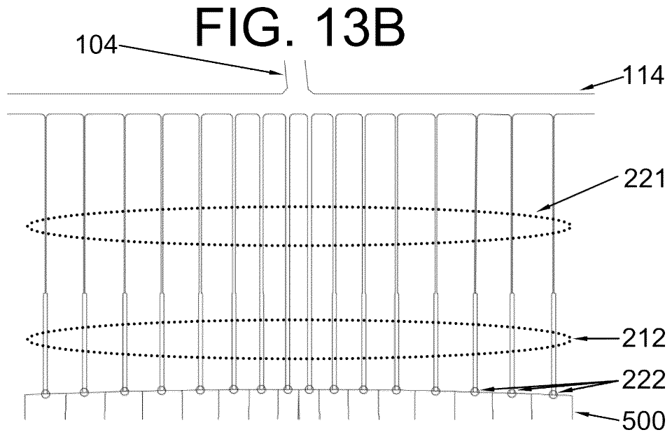

FIG. 13A shows one embodiment of the invention disclosed herein. FIG. 13A shows the left knee joint from the anterior/front view. In this embodiment, the air microfluidics channel networks module container 230 is fully integrated with the garment, meaning that there is no air microfluidics socket integrated with garment and that the air microfluidics chip is directly integrated with the garment. The container for the pneumatic module and the control center is also moved more proximally to the body's center of mass away from the knee joint; hence, at least one mini tubing integrated with garment 104 connects the said container with the rest of the fluidic components. Similarly, electrical pathways 403, 413 connect the EMG sensors and the IMU sensors to the control center for power and delivering biometric signals to the control center for processing and control via software. At least one mini channel for distribution of air 114 is used to supply the parallel air microfluidic pathways to induce sequential delays in pressurization and depressurization of the balloon actuators 500. The balloon actuators 500 are the same as the previous embodiment shown in FIG. 11. The key difference for this embodiment of the invention described herein is the use of air microfluidic module integrated with garment 230 which is boxed-in by dotted lines to indicate its location, which is shown in a close-up in FIG. 13B. Micro/mini channels to induce delay 221 have different channel cross-sectional area and are positioned parallel to each other. The difference in cross-sectional area of the micro/mini channels 221, as well as the parallel configuration of the said channels, allow for the concept of the path of least resistant through the principle of equivalent hydraulic resistance to induce sequential inflation and deflation of balloon actuators 500. Downstream from the micro/mini channels 221 are the mini channels for fluidic transportation 212 which have negligible equivalent hydraulic resistance; therefore, it is only used as a fluidic pathway for transportation. Certain micro/mini channels to induce delay 221 can be extended to become longer to increase equivalent hydraulic resistance if necessary, which means that in certain embodiments of the invention disclosed herein, some micro/mini channels to induce delay 221 fully or partially replaces the mini fluidic channels for fluidic transportation 212. In the embodiment shown by FIG. 13A and FIG. 13B of the invention described herein, the micro/mini channels to induce delay 221 and mini channels for fluidic transportation 212 are straight, but for certain embodiments of the invention described herein, the route for the fluidic channels 221, 212 can be of any shape including but not limited to serpentine shapes, curved shapes, square-wave shapes, sine-wave shapes and any spline shapes. The fluidic pathways connect to balloon actuators 500 via a connection point 222, which can be done by any method including but not limited to gluing, heat forming, chemical bonding, laminating and mechanically connecting.

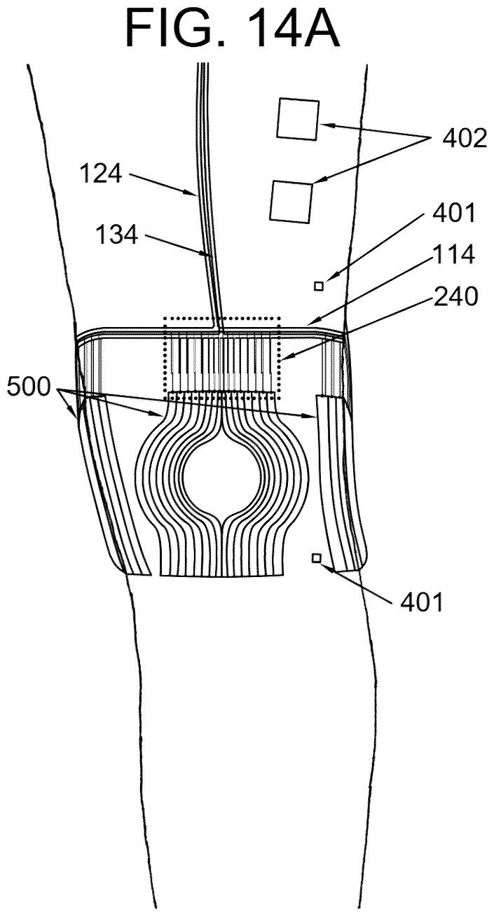

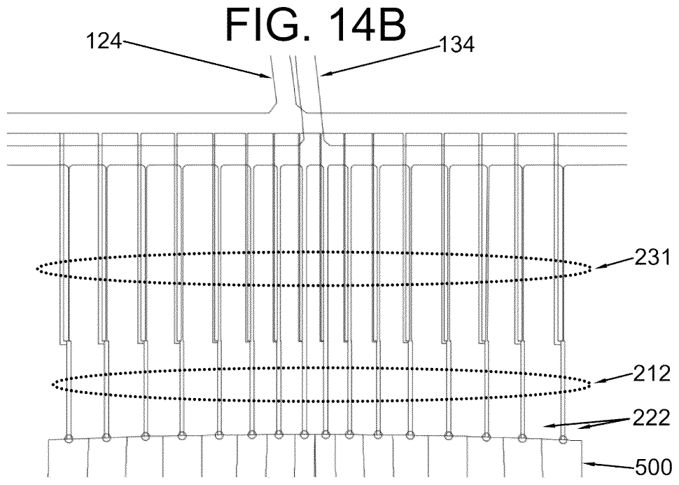

In another embodiment of the invention described herein as shown in FIG. 14A, the air microfluidic module integrated with garment 240 has separate channels with different cross-sectional areas for inflation and deflation for each fluidic pathway connected to each balloon actuator 500. Schematically, this can be referred to FIG. 5A and FIG. 5B. Separate channels for inflation and deflation allow for more robust active compression to create more complex massage effects. For example, consider two balloon actuators named “A” and “B”; balloon actuator “A” inflates before balloon actuator “B”, but balloon actuator “A” deflates after balloon actuator “B”; this sequence can be created by making the cross-sectional area of the inflation air microfluidic channel connecting to balloon actuator “A” smaller than that of balloon actuator “B”, and making the cross-sectional area of the deflation air microfluidic channel connecting to balloon actuator “A” bigger than that of balloon actuator “B”. Exactly how much smaller or bigger the cross-sectional channel areas of the air microfluidic channels depend on how much delay is required which in turn depends on the application and the anatomical portion of the human body the active compression apparel is addressing, as well as the locations of the balloon actuators. FIG. 14B shows the close-up of the air microfluidics module integrated with garment 240 in FIG. 14A. Two separate fluidic pathways are used 124, 134, one for inflation connected to at least one mini air pump, and another for deflation connected to at least one mini valve for deflation. The two fluidic pathways are completely separated and are only joined at the mini channels for fluidic transportation 212 or directly at the balloon actuators 500. The principle of equivalent hydraulic resistance and path of least resistant works beautifully here to demonstrate the robustness and scalability of the invention described herein. Furthermore, the EMG sensors 402 and IMU sensors 401 send signals to the control center wirelessly in the embodiment of the present invention shown in FIG. 14A; the said wireless technology for transmitting signal is preferably Bluetooth; however, other wireless technology may be used in certain embodiments of the invention described herein.

FIG. 15 shows yet another embodiment of the invention described herein. FIG. 15 shows an air microfluidics and air minifluidics enabled active compression device for the human left knee joint; this embodiment of the invention disclosed herein only differs from the one shown in FIG. 14A in the number and location of the balloon actuators 500. The balloon actuators 500 only exist on the anterior-medial and medial side of the left knee joint as shown in FIG. 15, whereas the balloon actuators 500 exist on the entire anterior, medial, lateral, and potentially posterior of the knee joint as shown in FIG. 14A. Therefore, the difference between the embodiments of the invention described herein shown in FIG. 15 and FIG. 14A indicates that the number, placement and the size of the balloon actuators may be tailored for each user and may be tailored based on application and the anatomical portion of the human body the active compression apparel is addressing.

The balloon actuators inflate due to the increase of the volume of air from the air flow generated by the mini air pump(s). At certain points of the inflation process, the balloon actuators whether made from elastomers or plastics would start to increase in internal pressure due to the increase of air density as the walls of the balloon actuators become taut and forms hoop stress. However, the pressure increase in the balloon actuators is not efficiently translated into compressional force onto the anatomical portions of the human body due to the isotropic property of the balloon actuators, meaning that the balloon actuators alone lack directional compression force. Therefore, garment encapsulating the balloon actuators are necessary to direct the compressional force inward onto the anatomical portions of the human body.

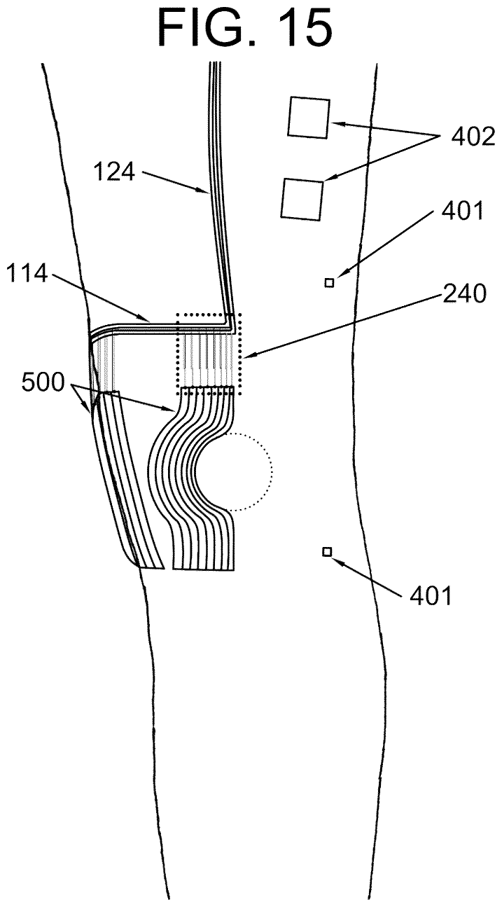

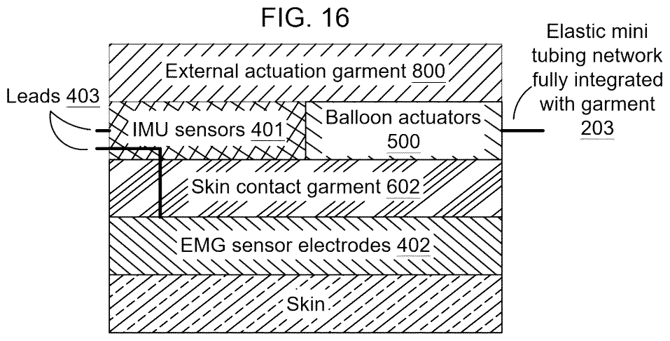

FIG. 16 shows the cross-sectional schematic of one embodiment of the garment for the active compression apparel of the invention described herein. It must be noted that the cross-sectional elements of FIG. 16 do not have to be placed exactly where they are shown in the schematic drawing, rather the drawing (FIG. 16) is only used to depict the approximate location and layer relationship between each element; certain variations and exceptions exist in any embodiments of the invention described herein. For example, in certain locations, the external actuation garment 800 may contact the skin contact garment 602 directly. The EMG sensors, and more specifically, the EMG sensor electrodes are situated directly on top of the human skin. At least one layer of skin contact garment 602 overlays the EMG sensor electrodes 402 and covers the skin. The purpose of the skin contact garment 602 includes but not limited to providing the user with comfort, sweat-wicking, and breathability. The skin contact garment 602 can be made from any fabric, polymers, or composites of fabric and polymers. The EMG sensor electrodes 402 can be attached to the skin contact garment 602 by sewing, lamination, gluing, mechanical bonding, chemical bonding, or any attachment method. The IMU sensors 401 and balloon actuators 500 are located above the skin contact garment 602. At least one external actuation garment 800 overlays the IMU sensor 401 and the balloon actuators 500. The external actuation garment 800 can be made from any fabric, polymers, or composites of fabric and polymers. In a preferred embodiment of the invention described herein, the IMU sensors 401 can be made waterproof by encapsulating it in a waterproof cover including but not limited to silicone, polydimethylsiloxane (PDMS), plastic, water repellent paint, and water repellent fabric. The IMU sensors 401 and balloon actuators 500 may be attached to the skin contact garment 602 and/or the external actuation garment 800 by sewing, laminating, gluing, mechanical bonding, chemical bonding, or any other attachment method. At least one electrical pathway leads 403 for transmitting power and signal to and from IMU sensors 401 and EMG sensor electrodes 402 are embedded within and/or under the external actuation garment 800 and above the skin contact garment 602. The electrical pathway leads 403 may be attached to the garments by sewing, laminating, gluing, mechanical bonding, chemical bonding, or any attachment method. The electrical pathway leads 403 may be connected to the IMU sensors 401 and EMG sensor electrodes 402 by soldering, mechanical connections, electrical conductive textile and any other electrical connection method. At least one elastic mini tubing network fully integrated with garment is embedded within and/or underneath the external actuation garment 800 and above the skin contact garment 602. The elastic mini tubing network integrated with garment 203 may be attached to the garments by sewing, laminating, gluing, mechanical bonding, chemical bonding and any other attachment method. The elastic tubing network fully integrated with garment 203 may be connected to balloon actuators 500 by gluing, laminating, mechanical bonding, chemical bonding, heating and any other connection method.

In certain embodiments of the invention disclosed herein, the balloon actuators 500 and elastic mini tubing network 203 do not require to be fully anchored within garments but rather allowed partially or completely free movement within the gap created by the external actuation garment 800 and the skin contact garment 602. Furthermore, in certain embodiments of the invention described herein, part of or the entire external actuation garment 800, IMU sensors 401 and EMG sensor electrodes 402 may be removed from the active compression apparel.

Various embodiments of the external actuation garment may be applied to the active compression apparel with the knee joint as an exemplary application. The importance of the external actuation garment cannot be understated. As mentioned earlier, the external actuation garment significantly increases the efficiency of the system of the invention described herein by providing directional compression onto the anatomical portions of the human body. In certain embodiments of the invention, the external actuation garment may be made out of one piece of fabric of the same material. However, the preferred embodiments of the invention described herein have external actuation garment made out of multiple pieces of fabric and out of different materials. The different materials allow for tailored compressional effect for different locations of the anatomical portions of the human body; these materials include but not limited to any combinations and ratios of nylon, polyester, spandex, silicone, polydimethylsiloxane (PDMS), and plastic. To further increase the directional compression efficiency, multiple independent and/or semi-independent external actuation garment may be used. Each independent and/or semi-independent external actuation garment is responsible for specific balloon actuators. Semi-independent external actuation garment means that the garments are detached at certain locations on the active compression apparel but are attached at other locations on the active compression apparel.

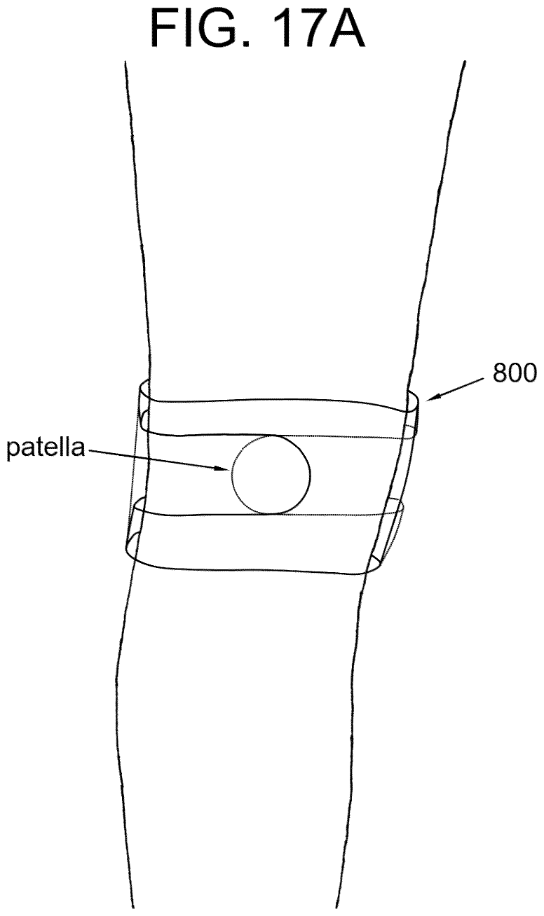

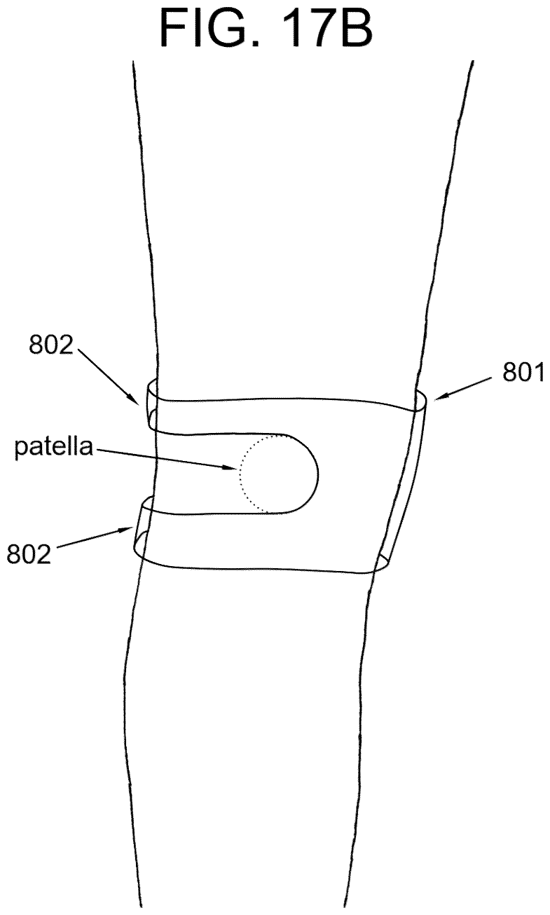

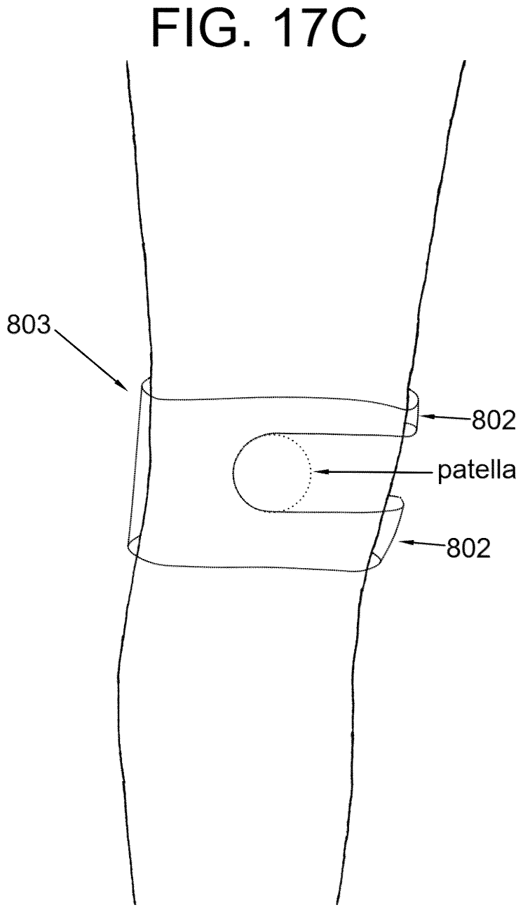

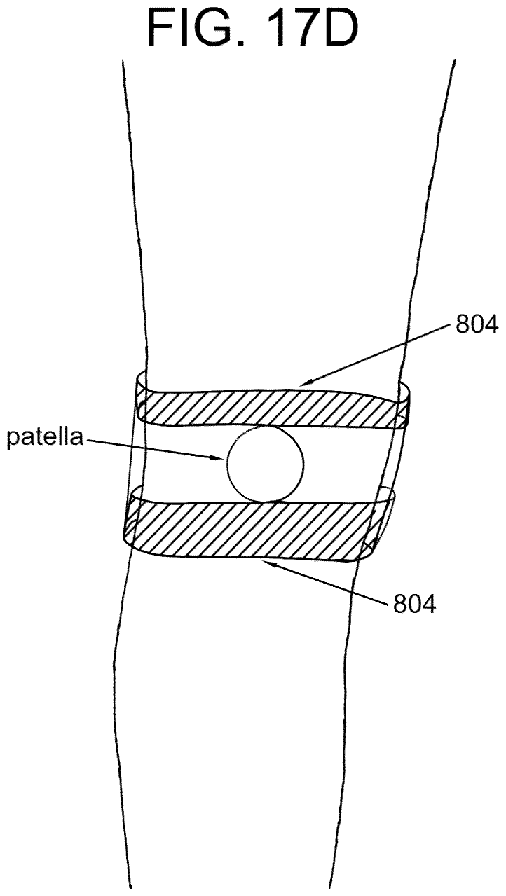

FIG. 17A shows one embodiment of the external actuation garment of the invention described herein. FIG. 17A shows a two-piece external actuation garment 800 for the human right knee joint from the anterior viewpoint. The principle of the multi-piece actuation garment 800 is that each piece of the actuation garment contains at least one actuation surface and at least one anchoring surface. The anchoring surface provides the leverage required for the actuation surface to apply appropriate compression force in the desired direction and location. Typically, the entire surface of the actuation garment 800 is also the actuation surface. In certain embodiments of the invention described herein, the anchoring surfaces and the actuation surfaces may overlap, meaning that certain surfaces may act as both an anchoring surface as well as an actuation surface for a particular piece of actuation garment. Furthermore, different pieces of actuation garment may overlay each other without unintentionally interfering with each other's compression forces. FIG. 17B and FIG. 17C more clearly indicate each piece of the two-piece external actuation garment 800 as shown in FIG. 17A and demonstrate the principle of the multi-piece actuation garment 800.

In FIG. 17B, the medial actuation garment 801 wraps around the knee joint just above and below the patella to the lateral/outside surface of the knee joint for anchoring purposes 802. When the balloon actuators inflate and apply pressure to everywhere the actuation garment exists, the anchors 802 on the lateral/outside surface of the knee joint prevents the medial actuation garment 801 from shifting and stretching beyond the pre-defined limits. It must be noted that certain compliance and creasing of the fabric is normal and fully expected, and the pressure exerted by the medial actuation garment 801 may not be equal at all locations. Furthermore, the anchors 802 may be a location and/or surface where the compressional force of a particular actuation garment 800 may be properly exerted and/or maximized. In other words, the anchoring location 802 provides the optimal leverage for the actuation garment 800 to exert compressional forces.

In FIG. 17C, the lateral actuation garment 803 wraps around the knee joint just above and below the patella to the medial/inside surface of the knee joint for anchoring purposes 802. The lateral actuation garment 803 differs from the medial actuation garment 802 regarding the surface it is addressing without any difference regarding functional mechanism. When the lateral actuation garment 803 and medial actuation garment 802 are overlaid on top of each other in no particular order, the multi-piece actuation garment 800 shown in FIG. 17A is formed. The two said garments 801 shown in FIG. 17B, and 803 shown in FIG. 17C have overlapping portions. For example, the actuation garment just above and below the patella are overlapping 804 as shown in FIG. 17D. In this particular embodiment of the present invention, the overlapping portions of the said garments 804 do not interfere with the overall function of the active compression apparel, which has the primary function of medial and lateral active compression, massage, and stabilization.

Furthermore, the compression forces would be minimized if balloon actuators either do not exist or do not inflate underneath the overlapping sections 804. It must be noted, the external actuation garment 800 is only part of the system for the present invention and only functions appropriately in conjunction with the rest of the elements of the system for the present invention. Also, the shape, the location, and the number of actuation garment 800 depend on the application and anatomical portions of the human body the active compression apparel is addressing.

FIG. 18 shows the actuation garment 800 of another embodiment of the invention described herein. The anterior/front view of the human right knee joint is shown in FIG. 18. Instead of two completely separate pieces of actuation garment as seen in FIG. 17A-D, the actuation garment 805, 806 are continuations of the overall/underlying apparel as seen by the lack of clear line separating the garments in FIG. 18. The underlying garment can be any fabric, but preferably stretch fabric that is commonly used in compression apparels. The actuation garments 805, 806 are preferably made out of fabric, polymer, or a composite of fabric and polymers that is tougher and less compliant than the base apparel to provide the directional compression forces.

FIG. 19 shows the actuation garment 800 of yet another embodiment of the invention described herein. The posterior/back view of the left knee joint of the human body is shown in FIG. 19. The actuation garment 800 in this configuration can be completely detached from the base apparel. The actuation garment 800 can be attached to the base apparel by attachment mechanism including but not limited to hook-and-loop fasteners, buckles, string laces, zippers, snaps and magnetic fasteners. Certain locations on the base apparel can be made to become attachment points for the actuation garment 800. FIG. 20 shows the actuation garment 800 of yet another embodiment of the present invention, where the actuation garment 800 is partially attached to the base apparel at one or multiple locations 808 with the rest of the said garment free to be attached at a preferred location on the base apparel. Certain locations 807 on the actuation garment 800 may be used for attaching to the base apparel.

### iv. Air Microfluidics and Minifluidics Systems, Examples and Implementations

The balloon actuators and various garments and sensors are the frontend of the present invention, meaning that they are the elements of the active compression apparel that directly contact the human body and apply various augmenting effects onto the anatomical portions of the human body the active compression apparel(s) is/are addressing. The backend is the conglomerate of fluidic and electronic hardware that must exist within the system of the present invention for the frontend to function. The most important hardware for the backend is the air microfluidics and air minifluidics components. An air microfluidics and air minifluidics system is realized when multiple air microfluidics and air minifluidics components are connected and assembled together.

Traditional pneumatic systems for controlling soft wearable robotics and wearable fluidic actuation systems, in general, are bulky due to many mechanical valves as well as fluidic pressure and/or flow transducers for controlling the actuation and sequencing of the frontend (i.e. balloon actuators, fluidic elastomer actuators, and McKibben artificial pneumatic muscle). The bulkiness introduces cumbersome factors such as less than desirable weight, size and footprint as well as undesirable aesthetics to the backend; hence the desirability of the overall system of soft wearable robotics and wearable fluidic actuation systems in general diminishes.

The advantage of air microfluidics and air minifluidics systems also resides in its capability of creating digital soft fluidic actuation, where multiple smaller balloon actuators replace single large balloon actuators. In other words, the compressional area force generated by a single large balloon actuator can be effectively mimicked by an array of tightly packed smaller balloon actuators. Furthermore, during human motion, the sequence of balloon actuator inflation and deflation is also important. Although sequential balloon actuator inflation and deflation can be achieved by multiple active valves, which is a “one design fits all” approach at the expense of bulkiness, active mechanical system reliability and complexity. Air microfluidics and air minifluidics systems can solve this problem by providing tailored sequential balloon actuator inflation and deflation with tailored air microfluidics and air minifluidics chips which have unique channel designs via equivalent hydraulic analogy concept introduced earlier. Each air microfluidics and air minifluidics chip is unique and can only provide one set of inflation and deflation sequence, which might be considered a disadvantage. However, in practical usage of active compression apparel, the compression sequence is generally tailored for each person without the need of changing over a certain period of time. Furthermore, in certain embodiments of the invention described herein, the air microfluidics and air minifluidics chip is detachable from the active compression apparel, which means that the compressional sequence effect can be changed easily. The inflation and deflation sequence of course only applies to transient response and given enough time, the pressure in all balloon actuators will equalize at steady state.

FIG. 21 shows one embodiment of the air microfluidics channel network module 200. The Air microfluidics chip 201 is detachable from the air microfluidics socket integrated with garment 202. A gasket 204 is cut to the shape of the air microfluidic socket integrated with garment 202 and sandwiched in between the air microfluidics chip 201 and the air microfluidics socket integrated with garment 202 to prevent leakage of air pressure from the vertical minifluidic pathway 205. The gasket 204 can be made from materials including but not limited to rubber, foam, silicone, plastic, leather and fiber. The method for attaching the gasket 204 to the surface of the air microfluidics chip 201 and/or air microfluidics socket integrated with garment 202 includes but not limited to tape, glue, mechanical fasteners, and epoxy. The gasket 204 may have any thickness, but preferably less than 0.5 mm. The vertical fluidic pathway 205 transports the air flow and pressure from the air microfluidics chip 201 and delivers to the air microfluidics socket integrated with garment 202 which then directs the airflow and air pressure into individual channels in the elastic mini channel network fully integrated with garment 203, which then passes onto each balloon actuators. In the embodiment of the invention described herein shown in FIG. 21, the air microfluidics chip 201 is attached to the air microfluidics socket integrated with garment 202 via magnets 206 for easy attachment and detachment. At least one mini tubing integrated with garment 104 connects the air microfluidics socket integrated with garment to the pneumatic module container.

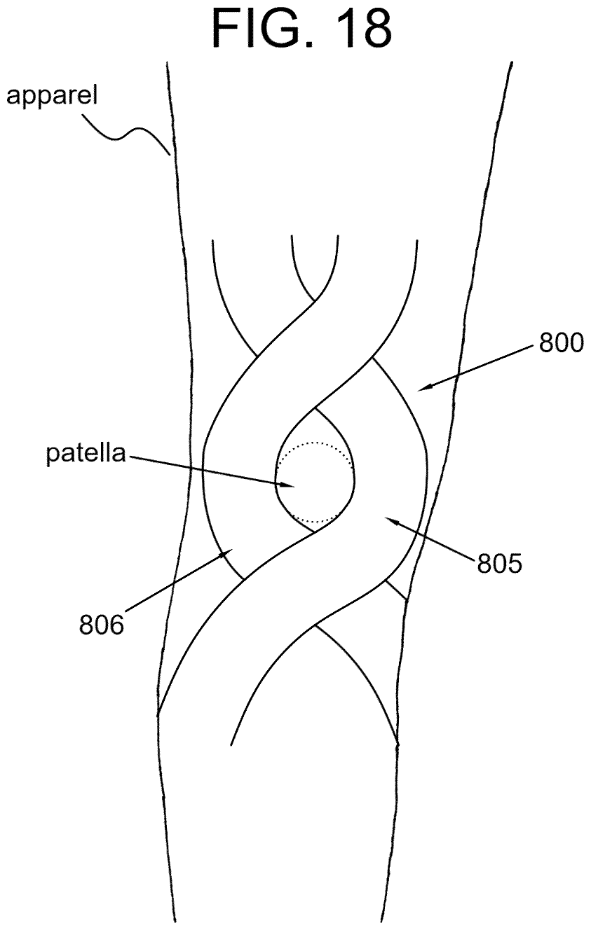

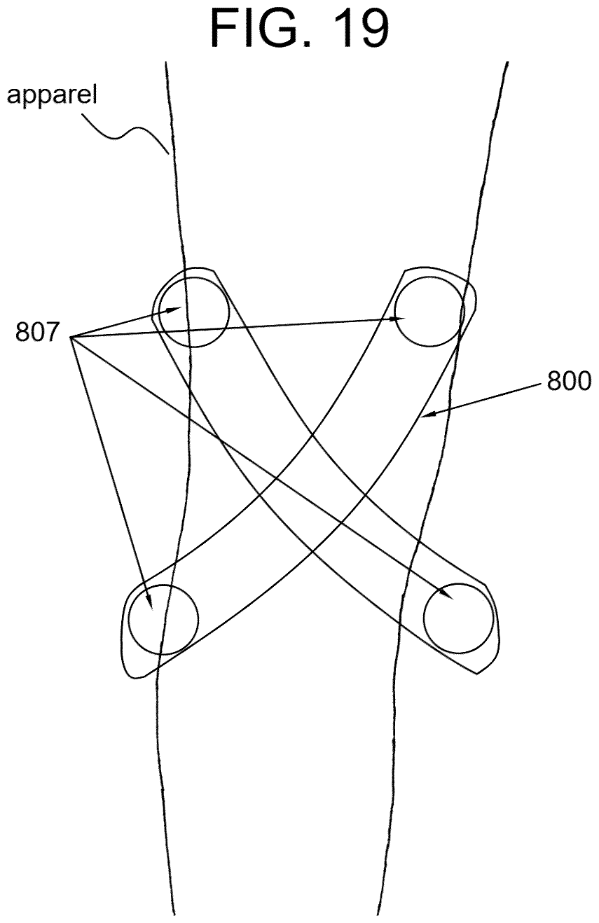

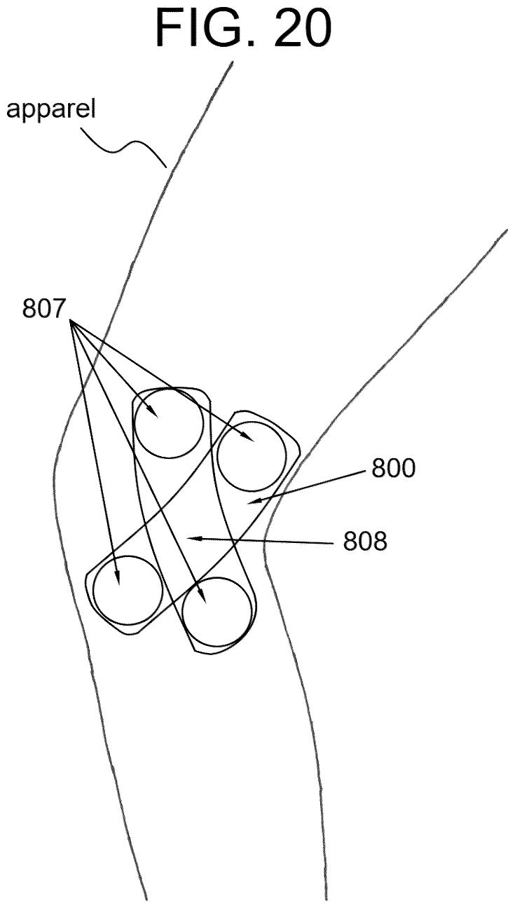

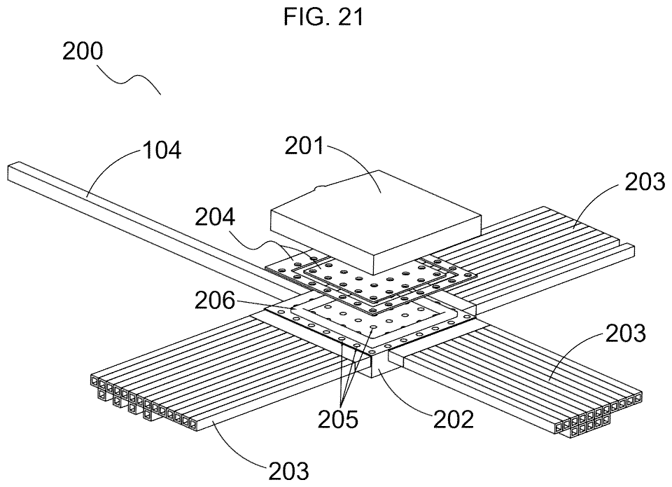

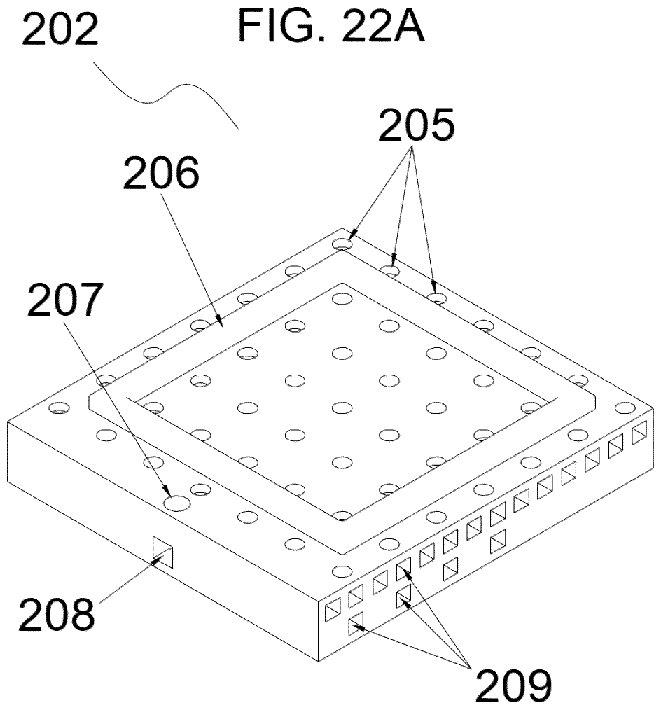

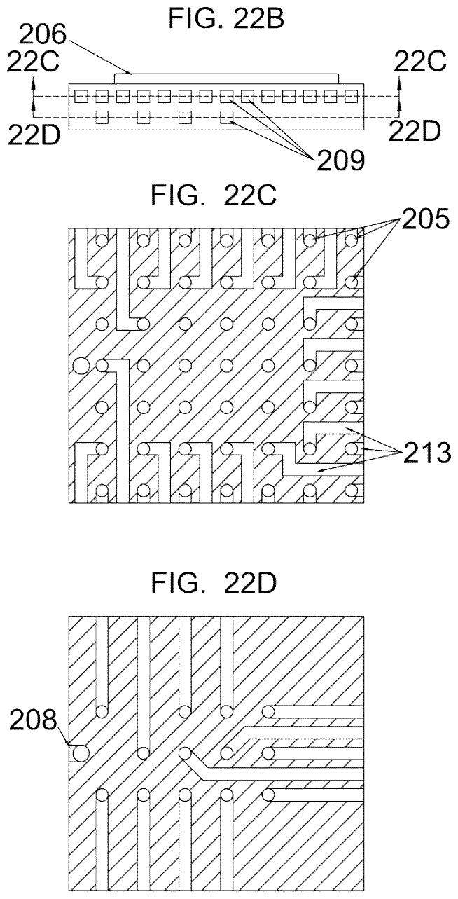

FIG. 22A, FIG. 22B, FIG. 22C, and FIG. 22D show the air microfluidics socket integrated with garment 202 by itself. The air microfluidics socket integrated with garment 202 acts as an air distribution manifold allowing for input air flow from the pneumatic module to reach the air microfluidics chip and output airflow from the air microfluidics chip to reach the balloon actuators. The material for making air microfluidics socket integrated with garment 202 includes but not limited to polydimethylsiloxane (PDMS), silicone, elastomers, plastics and metals. The vertical minifluidic pathways 205 connect to the vertical micro/mini channels in the detachable air microfluidics chip. The benefit of vertical minifluidic pathways 205 is the ease of connecting and aligning air microfluidic and air minifluidic channels. The output mini channels 209 connect directly to individual mini channels within the elastic mini channel network fully integrated with garment 203 shown in FIG. 21. Mini tubing integrated with garment connects directly with the input mini channel 208 and through the input vertical minifluidic pathway 207 to transport air from the pneumatic module to the air microfluidics chip. The magnet 206 is embedded within the air microfluidics socket integrated with garment 202 by gluing, mechanical fastening and/or any attachment methods. The magnet 206 protrudes above the top surface of the air microfluidics socket integrated with garment 202 as seen in FIG. 22A and FIG. 22B. The magnet ridge 206 from the air microfluidics socket integrated with garment 202 will mate with the magnetic groove on the air microfluidic chip for secure attachment. In other words, the air microfluidics socket integrated with garment 202 has the male part of the magnetic attachment, and the air microfluidics chip 201 has the female part of the magnetic attachment.

FIG. 22B shows the side view of the air microfluidics socket integrated with garment 202. The air microfluidics socket 202 has at least one layer of air distribution mini channel network. In the embodiment shown by FIG. 22A-D, there are two layers as seen in the cross-sectional view of FIG. 22C and FIG. 22D. The output mini channels 209 exist on three sides of the air microfluidics socket 202 with the last side dedicated to the input mini channel 208. As can be seen in FIG. 22C, the internal mini channels 213 connect the vertical minifluidic pathways 205 with the output mini channels 209. Due to the limited space within the air microfluidics socket 202, two layers are required to route the internal mini channels 213 to the sidewalls of the air microfluidics socket 202 without interfering with each other. It must be noted, that in certain embodiments of the invention described herein, one layer or multiple layers of internal mini channels 213 may be used for air microfluidics socket 202 depending on the application. The air microfluidic socket 202 can be integrated with a garment via methods including but not limited to sewing, gluing, laminating, taping, mechanical latching, and mechanical fastening.

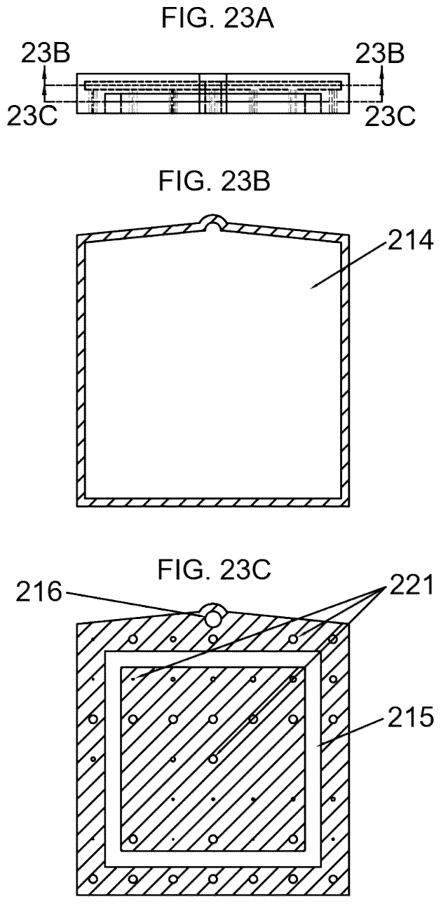

FIG. 23A, FIG. 23B, and FIG. 23C respectively shows the side view and two cross-sectional views of the air microfluidics chip 201 as seen in FIG. 21. A large reservoir chamber 214 as seen in the cross-sectional view (FIG. 23B) receives air from the air microfluidics socket via the vertical input minifluidic pathway 216 as seen in the cross-sectional view (FIG. 23C). The micro/mini channels to induce delay 221 and the magnetic groove 215 for attachment with the air microfluidics socket can be seen in FIG. 23C. It must be noted that although magnetic attachment is the preferred method for attaching the air microfluidics chip to the air microfluidics socket. Any other attachment method such as mechanical latches and mechanical fasteners may be used for other embodiments of the invention described herein.

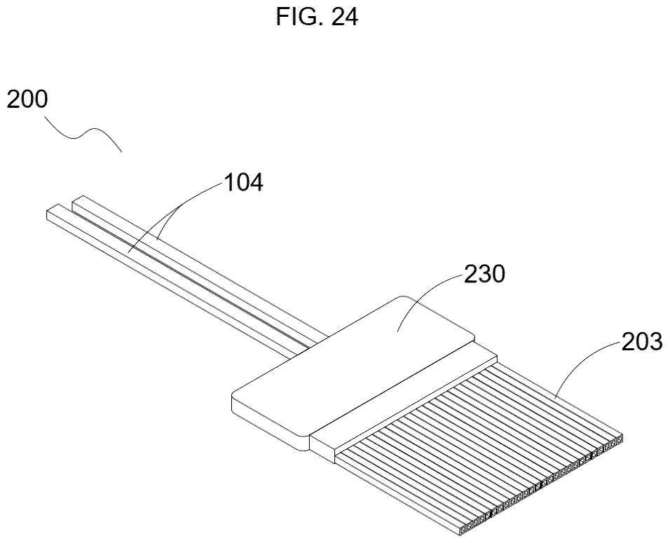

FIG. 24 shows a different embodiment of the air microfluidics channel networks module 200 which is the air microfluidics module integrated with garment 240 in the 2D schematic of FIG. 14A and FIG. 14B. Unlike the air microfluidics channel networks module 200 shown in FIG. 21, the air microfluidics channel networks module 200 shown in FIG. 24 is fully integrated with a garment, meaning that there is no air microfluidics socket and the air microfluidics chip 230 is fully integrated with the garment. The advantage of this system over the system shown in FIG. 21 is due to the fact that it is thinner as it has only one output elastic mini channel network layer 203 instead of the two shown in FIG. 21; hence, the thinner geometric profile allows for elastically compliable air microfluidics chips fully integrated with garment 230 that hugs the contour of any anatomical portions of the human body. Furthermore, the air microfluidics channel networks module 200 shown in FIG. 24 also allows for different inflation and deflation sequences by having separate micro/mini channels to induce delay for inflation and deflation of the balloon actuators; two separate mini tubing 104 where one is for inflation and the other for deflation are connected to the air microfluidics chip fully integrated with garment 230. FIG. 5A-B depicts the system shown in FIG. 24 on a schematic level.

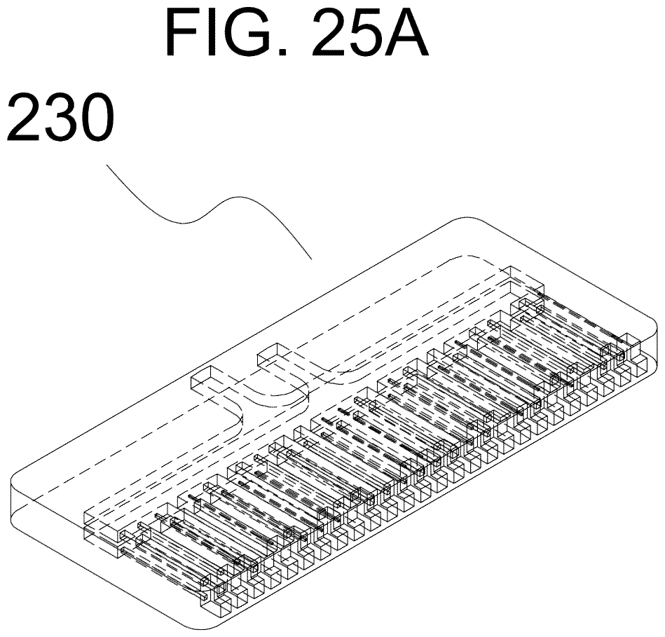

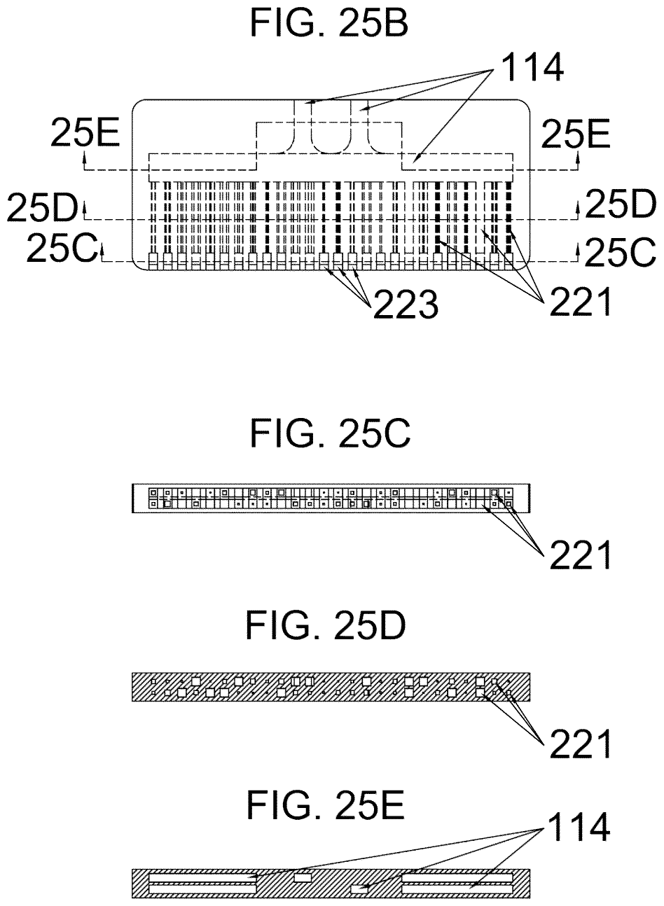

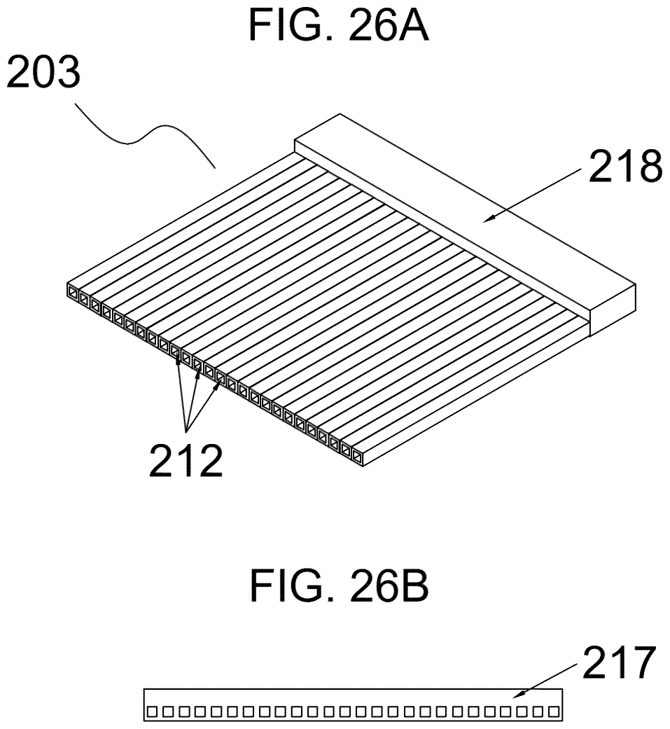

FIG. 25A-E shows various views of the of air microfluidics chip integrated with garment 230 by itself. FIG. 25A shows an isometric view of the air microfluidics chip integrated with garment 230. FIG. 25B shows the top view of the air microfluidics chip integrated with garment. Two mini channels for distribution of air 114 are used to supply and exhaust the parallel air microfluidic pathways to induce sequential delays in pressurization and depressurization of the balloon actuators. Each of the two mini channels for distribution of air 114 is connected to a mini tubing integrated with garment as seen in FIG. 24. The fluidic inflation pathway and the fluidic deflation pathway are completely separated until the exit mini channel 223 where the two micro/mini channels to induce delay 221 for inflating and deflating a single balloon actuator connect to a single mini channel 223 as seen in FIG. 25B; the said single mini channel 223 then connects with a particular mini channel within the elastic mini channel network fully integrated with garment 203 as seen in FIG. 24.

FIG. 25C shows the cross-sectional view at the exit mini channel location from FIG. 25B. The micro/mini channel to induce delay 221 for a single balloon actuator is stacked on top of each other vertically. It must be noted, although two separate networks of micro/mini channels to induce delay are used in the embodiment shown in FIG. 25A-C, more than two separate networks of micro/mini channels to induce delay may be used in other embodiments of the invention described herein.

FIG. 25D is the cross-sectional view showing the micro/mini channels to induce delay 221 of the air microfluidics chip integrated with garment 230 from FIG. 25B. FIG. 25E shows the cross-sectional view of the mini channels for distribution of air 114 of the air microfluidics chip integrated with garment 230 from FIG. 25B.

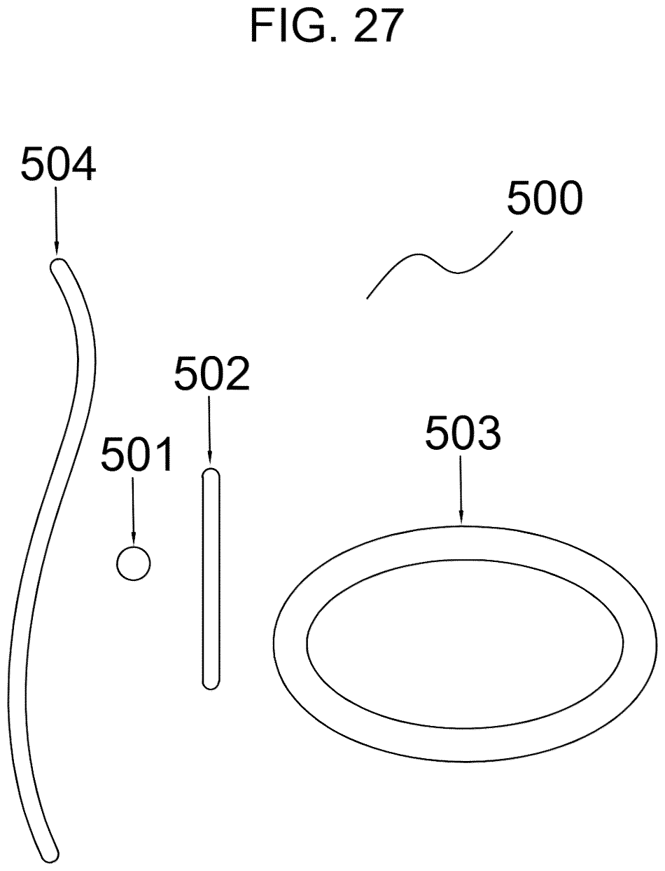

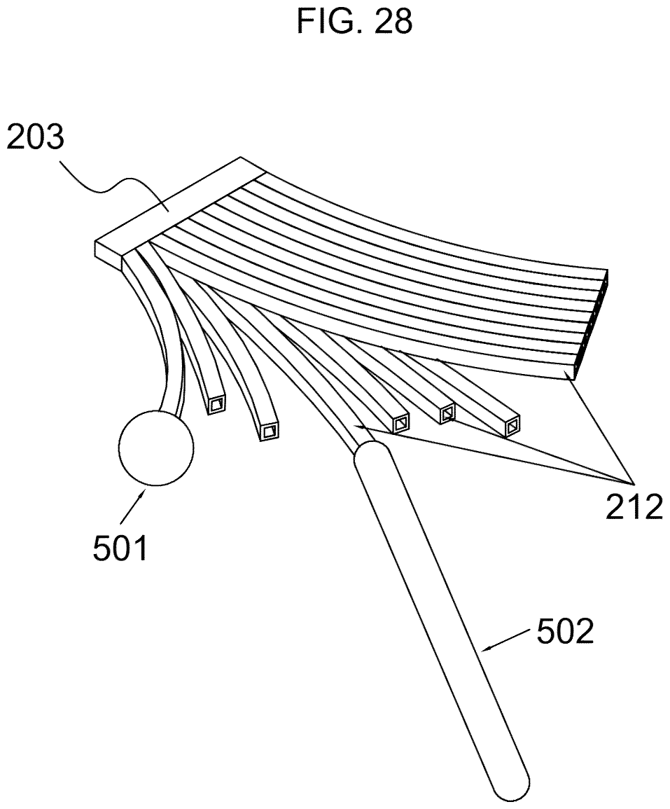

FIG. 26A-B shows the elastic mini channel network fully integrated with garment 203 for one embodiment of the invention described herein. FIG. 26A shows the isometric view of the elastic mini channel network fully integrated with garment 203. The elastic mini channels for fluidic transportation 212 and the attachment bar 218 make up the elastic mini channel network fully integrated with garment 203. The attachment bar 218 is used to connect to the air microfluidic chip integrated with garment or air microfluidics socket integrated with garment. FIG. 26B shows the back side of the elastic mini channel network fully integrated with garment 217, where its attachment bar 218 connects to the air microfluidics components as mentioned above. The elastic mini channel network fully integrated with garment 203 can be made from elastic materials including but not limited to silicone, PDMS (polydimethylsiloxane), rubber and elastomers. The fabrication process includes but not limited to 3D printing, injection moulding, extrusion moulding, and thermoforming. The connection between the elastic mini channel network fully integrated with garment may be permanent or detachable depending on the application and the type of active compression apparel. A permanent connection between the back side of the elastic mini channel network fully integrated with garment 217 and the above-mentioned air microfluidics components may be made using tape, plasma bonding, adhesives, thermal bonding and any other appropriate permanent bonding method. The detachable connection between the back side of the elastic mini channel network fully integrated with garment 217 and the above-mentioned air microfluidics components may be made using mechanical fasteners, magnets, and any other appropriate detachable connection method.

Furthermore, to increase the permanent bonding strength between the back side of the elastic mini channel network fully integrated with garment 217 and the above mentioned air microfluidics components, adhesives and bonding material including but not limited to glue, silicone, and tape can be applied around the outer seam of the bonding surface in a welding fashion. Certain embodiments of the invention described herein can use a one-piece fabrication process via 3D printing, meaning that the entire air microfluidics channel network module is fabricated as one piece with no seams or connection points. The preferred 3D printing process is stereolithography; however, other 3D printing processes may be used in certain embodiments of the invention described herein.

FIG. 27 shows four different exemplary shapes of the balloon actuators. It must be noted that the balloon actuators can be of any shape and size depending on the application and the anatomical portion of the human body the active compression apparel is addressing. The spherical balloon actuator 501 is typically the smallest balloon actuator when compared to other balloon actuators. The advantage of spherical balloon actuator 501 lies in its ability to apply concentrated point forces that can mimic an area compression force when multiple balloon actuators 501 are placed in an array. Furthermore, arrays of balloon actuators 501 can be placed around joints for active compression as they do not impede joint movement due to the small size of individual balloon actuators 501. The elongated balloon actuators 502 is the most versatile balloon actuator as it can fit any anatomical portions of the human body by varying its length. Furthermore, the elongated balloon actuators 502 may apply torque in addition to compression when inflated. The donut-shaped balloon actuators 503 surround anatomical portions of the human body, preferably around limbs and preferably applying compression to muscles. The irregular-shaped balloon actuator 504 is robust and can address any anatomical portions of the human body. The balloon actuators 500 can be made from any material that has elasticity; the preferred material for making balloon actuators is thin plastic membrane and silicone elastomers. The preferred method for making balloon actuators is through heat sealing at least two plastic sheets or through the balloon manufacturing process, which is a type of moulding process.

FIG. 28 shows the elastic mini channel network fully integrated with garment 203 with two mini channels for fluidic transportation 212 connecting to two balloon actuators 501, 502. The mini channels for fluidic transportation 212 can be made into single combined conduits or separated into single tubing depending on the application and the anatomical portions of the human body the active compression apparel is addressing. The balloon actuators may be connected to the mini channels for fluidic transportation 212 via heat sealing, tape, glue, or any appropriate connection methods. Also, non-permanent connections between balloon actuators and mini channels for fluidic transportation 212 including but not limited to fittings may also be used for certain embodiments of the invention described herein.

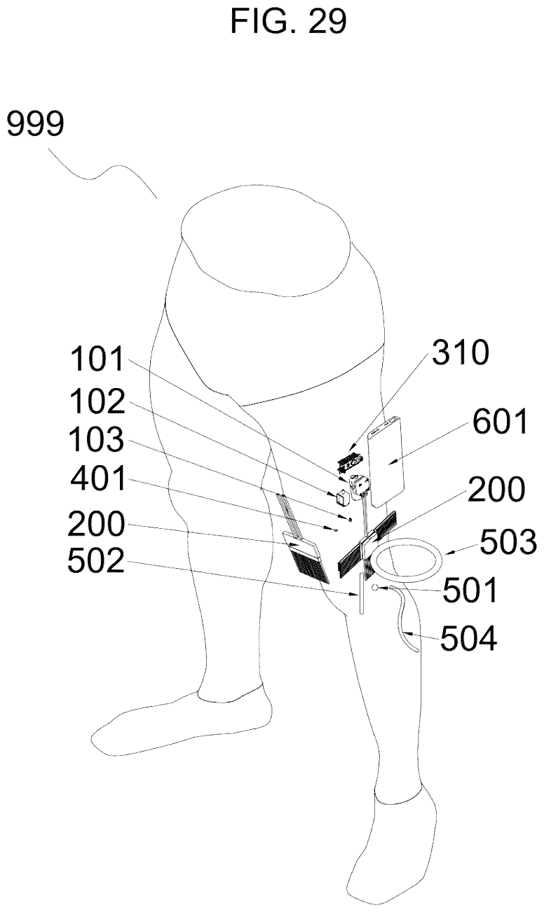

FIG. 29 shows the size and scale of many hardware components of the invention described herein with comparison to the human lower extremity 999. Please note that the components shown in FIG. 29 are only for exemplary purpose and only represent certain embodiments of the invention described herein. The size of components for other embodiments of the present invention may differ. Off-the-shelf components are used to represent the scale of certain classes of components. A person skilled in the art may choose other components within the same class depending on the applications and the anatomical portion of the human body the active compression apparel is addressing. The components in FIG. 29 are used for embodiments of the present invention addressing the lower extremity of the human body. The battery pack 601 is a 10,000 mAh portable battery from SAMSUNG (EB-P1100BSEGUS). The microcontroller 310 is an Arduino Nano, which is an opensource microcontroller. The mini air pump 101 is a 22K series miniature diaphragm pump from BOXER. The mini valve 102 is a normally open 2-port 1-way solenoid valve that can be purchased from many different vendors such as Amazon and AliExpress. The air pressure sensor 103 is a MPR series pressure sensor from Honeywell. The IMU sensor 401 is a 9-axis IMU from TDK (ICM-20948). Two different embodiments of the air microfluidics channel networks module 200 from FIG. 21 and FIG. 24 are also included and placed near the locations where they may be placed for active compression apparels of the knee joint. The size and scale of the balloon actuators 501, 502, 503, 504 are shown as they are placed next to the knee joint in FIG. 29.

### v. Software and Control

In the above sections, the hardware, operating principles, and exemplary applications and preferred embodiments are shown and discussed in detail in ways that a person skilled in the art can faithfully reproduce any embodiments of the invention described herein. However, hardware alone without software cannot make the air microfluidics and air minifluidics enabled active compression garment function. This section shows and discusses various embodiments and examples of the software and control strategies required to enable the invention described herein function appropriately.

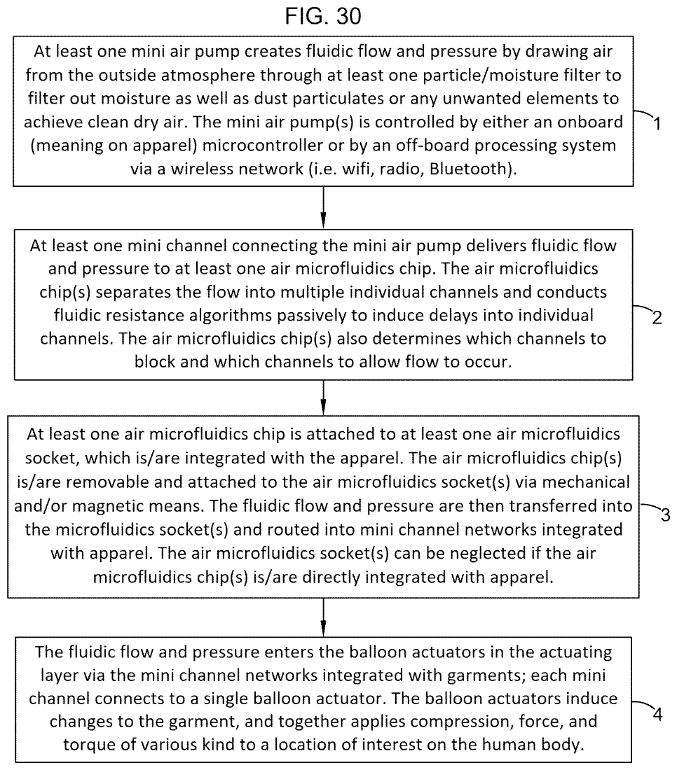

FIG. 30 is a flow chart depicting the series of events for the fluidic system of one embodiment of the present invention required for the inflation/pressurization of the balloon actuators. As mentioned by block 1, air is drawn from the external atmosphere by at least one mini air pump into the internal fluidic system. Particle/moisture filters are used to removed unwanted particles such as dust particles to avoid clogging the mini channels and micro channels. It must be mentioned that in certain embodiments of the invention described herein, an internal tank may be used to hold air and used to inflate the balloon actuators before drawing air from the external environment for maximizing the efficiency of the system as shown by FIG. 3. The mini air pump(s) can be controlled by an onboard microcontroller such as an Arduino, or by an off-board processing system via a wireless network. For instance, all the processing and software algorithm can be done via an application software on a smartphone, a smartwatch, and/or a mobile computing device. As mentioned by block 2, the air drawn from the mini air pump(s) enters mini channels which deliver the air into air microfluidics chip(s), where the airflow is separated into individual channels, and the passive fluidic resistance algorithms via the concept of equivalent hydraulic resistance are performed. The passive fluidic resistance algorithms induce passive delays in the inflation and deflation of the balloon actuators. As mentioned by block 3, the divided airflows that have undergone fluidic resistance algorithm exit the air microfluidics chip(s) or air microfluidics socket(s) via individual mini channels, which act as final delivery fluidic pathways to the balloon actuators. Lastly, as mentioned by block 4, the balloon actuators inflate in a pre-defined sequences which induce changes to the garment, and applies compression to an anatomical portion of the human body for a variety of effects including but not limited to minimizing the risks of injuries, massages to reduce pain, increased proprioception, increased bodily fluid circulation, better ergonomics, and general comfort.

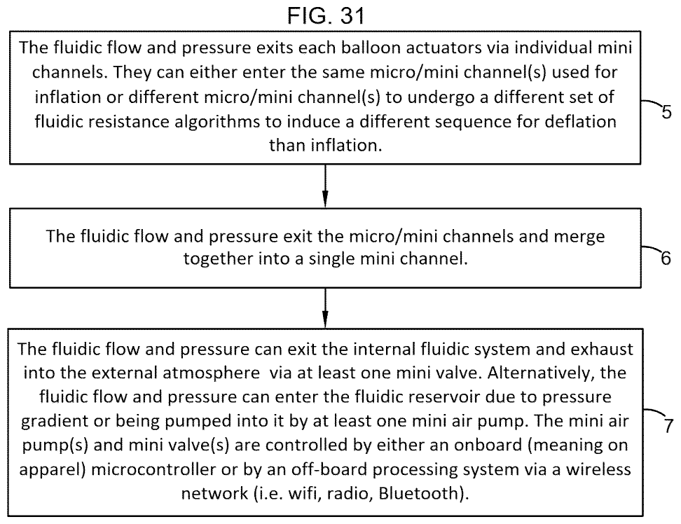

FIG. 31 is a flow chart depicting the series of events for the fluidic system of one embodiment of the present invention required for the deflation/depressurization of the balloon actuators. As mentioned by block 5, each balloon actuator deflates by exhausting air through the same individual mini channel as the inflation sequence. The air then enters the micro/mini channels to undergo fluidic resistance algorithm for induced sequential delay in the depressurization/deflation of the balloon actuators; these micro/mini channels can be the same ones as the inflation sequence or different ones. As mentioned by block 6, the fluidic flow and pressure exit the micro/mini channels and merge together into a single mini channel. Lastly, as mentioned by block 7, the fluidic flow and pressure are exhausted either into the external atmosphere and/or a fluidic reservoir tank. In both cases, a mini valve is required, and in the case of exhausting into a fluidic reservoir tank, mini air pumps(s) may be implemented. The mini valve(s) and the mini air pump(s) can be controlled by an onboard microcontroller such as an Arduino, or by an off-board processing system via a wireless network.

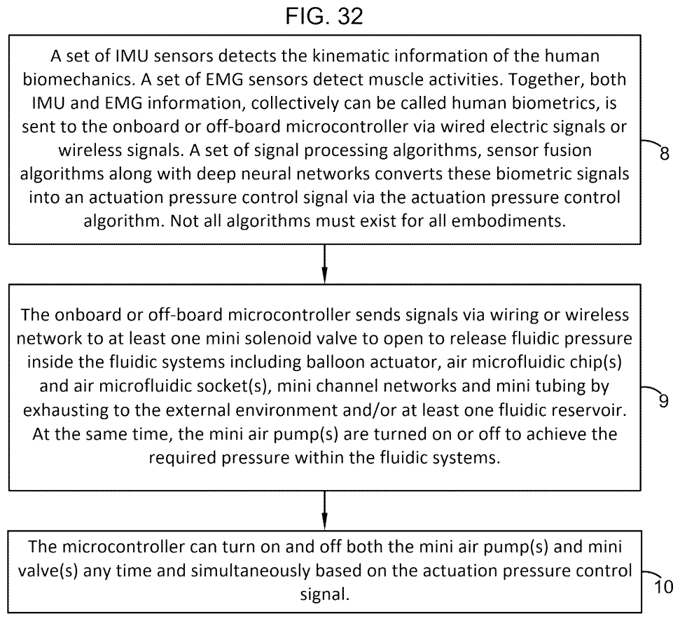

FIG. 32 is a flow chart depicting a series of events for the sensor systems and the control system of one embodiment of the invention described herein. The control system is used to activate the mini air pump(s) and the mini valve(s). The air microfluidics module is entirely passive, which is the reason that this system can achieve sequential inflation and deflation without having large amount of power-consuming and bulky components. As mentioned by block 8, biometric signals such as kinematic motion information from IMU sensors and muscle activity information from EMG sensors are sent to the onboard or off-board microcontroller via signal pathways including but not limited electrical wiring or wireless signal transmission methods. A set of signal processing algorithms, sensor fusion algorithms along with deep neural networks converts these biometric signals into an actuation pressure control signal via the actuation pressure control algorithm. As mentioned by block 9, the control algorithm will continuously activate the mini air pumps and the mini valves to satisfy the outputs of the actuation pressure control algorithm which is air pressure. As mentioned by block 10, the microcontroller can turn on and off both the mini air pump(s) and mini valves(s) any time and simultaneously based on the actuation pressure control signal.

It must be noted, the digital soft fluidic actuation method via air microfluidics and air minifluidics allows for longevity of the active components such as the mini valve(s) and mini air pump(s). The reason is that the inflation and deflation of the balloon actuators can be timed and each balloon actuator can be considered to be either on or off. A higher area compression force is a result of having more balloon actuators turned on. Hence, knowing how long it takes to turn on a balloon actuator allows for precise control of the system. The pressure sensor(s) may be used for redundancy checks and safety.

Since most of inflation and deflation sequencing is controlled by the passive air microfluidics and air minifluidics modules, the control can be achieved by open-loop control strategies or closed-loop feedback control strategies such as on-off control, PI control, and PID control. However, other controllers including but not limited to feedforward control, adaptive control, and optimal control may still be implemented depending on the applications.

The software can be written in any programming language. The sensor fusion algorithm combines the data from EMG sensors and IMU sensors and outputs a reference signal of the pressure required in the balloon actuators and/or the amount of time the mini air pump(s) need to pump to achieve the desired pressure in the balloon actuators. The signal processing algorithm increases the signal to noise ratio of the sensor signals and converts the EMG sensors into the correct format (i.e. full wave rectified, average EMG, RMS EMG, integrated EMG, frequency domain EMG). The artificial neural network functions in conjunction with a sensor fusion algorithm to provide the actuation signal. For example, the artificial neural network can determine the movement, motion, and the lifestyle pattern of the user to tailor the active compression. Over time, the artificial neural network can become better through learning the movement patterns of the user.

For certain embodiments of the invention described herein, Sensor calibration would be required at the beginning of each use session. For instance, the IMU sensor must establish an initial frame of reference, preferably having the vertical axis aligned with the direction of gravitational pull or in an appropriate position depending on applications. Furthermore, The EMG sensor would also preferably be calibrated at the beginning of each use session. For instance, at least a two-point calibration would be required to determine the resting state and the maximum exertion state of the muscle groups surrounding the anatomical portion of the human body the active compression apparel is addressing. For certain embodiments of the invention described herein, recalibration during a use session may be required to reduce drift in sensors.

For certain embodiments of the invention described herein, mobile application(s) on a smartphone, smartwatch, and similar mobile computing devices may provide a graphical user interface which the user may manually control the air microfluidics and air minifluidics enabled active compression apparel or set parameters that allow automated control of the said active compression apparel. The mobile application(s) may also provide crucial system information including but not limited to battery life remaining, whether maintenance is required, parts to be replaced, and the pressure reading from the pressure sensor(s). Furthermore, the mobile application(s) may also download various software updates for the control center.

It must be noted that certain embodiments may have all of the software described here, whereas certain other embodiments may have only part of the software described herein. A person skilled in the art can faithfully reproduce the software and control strategies of any of the embodiments of the invention described herein.

The invention is contemplated for use in association with air microfluidics and air minifluidics enabled active compression devices, garments, and methods to afford increased advantageous utilities in association with same. The invention, however, is not so limited, and can be readily used with other items to afford various advantageous utilities within the scope of the invention. Other embodiments, which fall within the scope of the invention, may be provided.

The foregoing description has been presented for the purpose of illustration and is not intended to be exhaustive or to limit the invention to the precise form disclosed.

Naturally, in view of the teachings and disclosures herein, persons having ordinary skill in the art may appreciate that alternate designs and/or embodiments of the invention may be possible (e.g., with substitution of one or more components for others, with alternate configurations of components, etc). Although some of the components, relations, configurations, and/or steps according to the invention are not specifically referenced and/or depicted in association with one another, they may be used, and/or adapted for use, in association therewith. All of the aforementioned and various other structures, configurations, relationships, utilities, any which may be depicted and/or based hereon, and the like may be, but are not necessarily, incorporated into and/or achieved by the invention. Any one or more of the aforementioned and/or depicted structures, configurations, relationships, utilities and the like may be implemented in and/or by the invention, on their own, and/or without reference, regard or likewise implementation of any of the other aforementioned structures, configurations, relationships, utilities and the like, in various permutations and combinations, as will be readily apparent to those skilled in the art, without departing from the pith, marrow, and spirit of the disclosed invention.

Other modifications and alterations may be used in the design, manufacture, and/or implementation of other embodiments according to the present invention without departing from the spirit and scope of the invention, which is limited only by the claims of this patent application and any divisional and/or continuation applications stemming from this patent application.

1. A wearable air microfluidics and minifluidics device, for use with one or more garments worn by a user, wherein the device comprises:
(a) balloon actuators, configured for integration with the one or more garments, and configured to apply one or more predetermined forces to one or more anatomical portions of the user's body when inflated with gas; wherein the forces comprise active compression and/or augmenting forces;
(b) an air channel module comprising one or more small-scale air channels in fluid communication with the balloon actuators; wherein the small-scale air channels comprise air micro channels and/or air mini channels;
(c) a pneumatic module in fluid communication via the small-scale air channels with the balloon actuators; wherein the pneumatic module, when activated, induces flow of the gas under pressure, through the small-scale air channels, to the balloon actuators;
(d) one or more sensors, configured for integration with the one or more garments, and configured to generate signals based on biometric data and/or user motion detected at the garment; and
(e) a control module that selectively, depending upon the signals from the sensors, activates the pneumatic module to inflate and deflate the balloon actuators, to apply the predetermined forces to the anatomical portions of the user's body, based on the biometric data and/or user motion.

2. The device according to claim 1, wherein the air channel module, the pneumatic module, and the control module are configured for secure attachment to the garments.

3. The device according to claim 1, wherein the air channel module is configured to use equivalent hydraulic resistance and to induce passive delays in pressurization and depressurization of the balloon actuators.

4. (canceled)

5. (canceled)

6. (canceled)

7. The device according to claim 1, wherein at least a portion of the air channel module is configured for selectively removable integration with the one or more garments.

8. (canceled)

9. The device according to claim 1, wherein the small-scale air channels are combined into a network; and wherein each respective one of the small-scale air channels is elastic, flexible, or rigid.

10. The device according to claim 1, wherein the air channel module further comprises: (a) one or more air microfluidics chips, and (b) an elastic mini channel network that is configured for integration with the one or more garments; wherein the small-scale air channels are embodied in both the air microfluidics chips and the elastic mini channel network; and wherein at least some of the small-scale air channels embodied in the elastic mini channel network are said air mini channels and are elastic.

11. The device according to claim 10, wherein the air channel module further comprises an air microfluidics socket that is adapted to receive at least a first selected one of the air microfluidics chips in fluid communication with the elastic mini channel network.

12. (canceled)

13. (canceled)

14. (canceled)

15. (canceled)

16. (canceled)

17. The device according to claim 1, wherein at least a portion of the pneumatic module is configured for selectively removable integration with the one or more garments.

18. (canceled)

19. The device according to claim 1, wherein the pneumatic module comprises a fluidic reservoir, and the pneumatic module draws gas from the fluidic reservoir.

20. (canceled)

21. The device according to claim 1, wherein the pneumatic module comprises a mini/micro air pump and one or more mini/micro valves, configured for integration with the one or more garments, in fluid communication with the small-scale air channels.

22. (canceled)

23. The device according to claim 1, wherein the control module operatively executes a sensor fusion subroutine to reconcile and combine the signals from the sensors into a substantially complete dataset of the biometric data and/or user motion that was detected at the garment.

24. The device according to claim 1, wherein the control module operatively executes an artificial neural network subroutine to determine user motion patterns of the user's body and/or said anatomical portions of the user's body.

25. The device according to claim 1, wherein the control module comprises physical hardware, with at least some of the physical hardware configured for integration onboard the one or more garments.

26. (canceled)

27. (canceled)

28. (canceled)

29. (canceled)

30. (canceled)

31. (canceled)

32. (canceled)

33. (canceled)

34. (canceled)

35. The device according to claim 1, wherein the device is adapted for use with garments which have an outer garment layer, and wherein each of the balloon actuators is configured to be positioned between the outer garment layer and the user's skin.

36. (canceled)

37. An air microfluidics and minifluidics garment, adapted to be worn by a user, wherein the garment comprises:
(a) one or more outer garment layers;
(b) balloon actuators, positioned between the outer garment layers and the user's skin, that apply one or more predetermined forces to one or more parts of the user's body when inflated with gas; wherein the forces comprise active compression and/or augmenting forces;
(c) an air channel module comprising one or more small-scale air channels in fluid communication with the balloon actuators; wherein the small-scale air channels comprise air micro channels and/or air mini channels;
(d) a pneumatic module in fluid communication via the small-scale air channels with the balloon actuators; wherein the pneumatic module, when activated, induces flow of the gas under pressure, through the small-scale air channels, to the balloon actuators;
(e) one or more sensors that receive signals from the garment based on biometric data and/or motion of the user; and
(f) a control module that selectively, depending upon the signals from the sensors, activates the pneumatic module to inflate and deflate the balloon actuators, to apply the predetermined forces to the parts of the user's body, based on the biometric data and/or motion of the user.

38. The garment according to claim 37, further comprising at least one inner garment layer that contacts the user's skin, wherein the balloon actuators are positioned between the outer garment layers and the inner garment layer, and wherein the air channel module, the pneumatic module, the sensors, and the control module are securely attached to the outer garment layers and/or to the inner garment layer.

39. (canceled)

40. (canceled)

41. (canceled)

42. An air microfluidics and minifluidics method for applying one or more predetermined forces to one or more anatomical portions of a user's body, wherein the method comprises:
(a) a detection step of using one or more sensors, configured for integration with one or more garments that are adapted to be worn by a user, to generate signals based on biometric data and/or user motion detected at the one or more garments;
(b) a control step of using a control module to selectively, depending upon the signals from the sensors, activate a pneumatic module to induce flow of gas under pressure, through small-scale air channels of an air channel module;
(c) an air channel step of using the small-scale air channels of the air channel module in fluid communication with balloon actuators to convey said flow of said gas under pressure to the balloon actuators; wherein before the air channel step, the small-scale air channels are provided in the form of air micro channels and/or air mini channels; and
(d) a balloon actuation step of inflating and deflating balloon actuators, configured for integration with the one or more garments, to apply the predetermined forces to the anatomical portions of the user's body, based on the biometric data and/or user motion; wherein the forces comprise active compression and/or augmenting forces.

43. The method according to claim 42, wherein in the air channel step, the air channel module uses equivalent hydraulic resistance and induces passive delays in pressurization and depressurization of the balloon actuators in the balloon actuation step.

44. (canceled)

45. (canceled)

46. (canceled)

47. The method according to claim 42, wherein in the control step, the control module operatively executes an actuation subroutine that, in selectively activating the pneumatic module, controls a mini/micro air pump and one or more mini/micro valves of the pneumatic module that are configured for integration with the one or more garments, in fluid communication with the small-scale air channels.

48. The method according to claim 42, wherein in the control step, one or more software components are, at least partially, operatively executed on a portable computing device that is off-board the one or more garments, and the software components enable the user, at least partially, to manually control selective activation of the pneumatic module, to input predetermined settings for automated control of the pneumatic module and/or the balloon actuators, to track performance and information regarding the pneumatic module and/or the balloon actuators, and/or to update the software components.

49. (canceled)

50. (canceled)

51. (canceled)
